(19) United States
(12) Patent Application Publication
(10) Pub. No.: US 2026/0221216 A1
(43) Pub. Date: Jul. 30, 2026

(54) GENERATING TRANSCRIPTOMIC BENCHMARKS FOR TRANSCRIPTOMICS MACHINE LEARNING MODELS UTILIZING A STRUCTURAL INTEGRITY METRIC

(71) Applicant: Recursion Pharmaceuticals, Inc., Salt Lake City, UT (US)

(72) Inventors: Alisandra Kaye DENTON, Montreal (CA); Finagnon Marc-Rolland Emmanuel NOUTAHI, Montreal (CA); Ihab BENDIDI, Paris (FR); Kian Runnels KENYON-DEAN, Toronto (CA); Shawn Tamajka WHITFIELD, Montreal (CA); Yassir EL MESBAHI, Montreal (CA)

(21) Appl. No.: 19/041,384

(22) Filed: Jan. 30, 2025

(51) Int. Cl. G16B 15/00; G16B 40/00

(52) U.S. Cl. CPC G16B 15/00; G16B 40/00

(57) ABSTRACT

The present disclosure relates to systems, methods, and non-transitory computer-readable media for utilizing transcriptomic evaluation metrics to generate a transcriptomic benchmark for a transcriptomics machine learning model. In some embodiments, the disclosed systems can generate embeddings from observed transcriptomic profiles resulting from perturbations. By utilizing the embeddings, the disclosed systems can generate an evaluation framework of transcriptomic evaluation metrics, which includes a structural integrity metric. For instance, to generate the structural integrity metric, the disclosed systems may: reconstruct predicted transcriptomic profiles from the embeddings; generate adjusted transcriptomic profiles by applying control profiles; determine a structural distance between adjusted observed transcriptomic profiles and adjusted predicted transcriptomic profiles; and compare the structural distance to a determined threshold structural distance. Upon generating the structural integrity metric and other transcriptomic evaluation metrics within the evaluation framework, the disclosed systems can combine the transcriptomic evaluation metrics to generate the transcriptomic benchmark.

### BACKGROUND

In the field of deep learning, recent years have seen significant developments in exploring relationships among genes, compounds, and their interactions in living organisms. For instance, existing deep learning models have been developed to predict protein folding from their sequences, to understand binding dynamics, or to identify biological relationships through analysis of large-scale microscopy imaging data of perturbed cells. While conventional systems have successfully employed various modalities of biological data for perturbation analysis, these conventional systems often underutilize transcriptomics-which provides detailed insights into cellular states. Further, although there have been some developments in transcriptomics sequencing techniques and datasets focused on perturbations, conventional systems nevertheless exhibit a number of deficiencies or drawbacks, particularly relating to functionality, accuracy, and efficiency.

These along with additional problems and issues exist with regard to conventional systems.

### BRIEF SUMMARY

This disclosure describes one or more embodiments of systems, methods, and non-transitory computer-readable storage media that provide benefits and/or solve one or more of the foregoing and other problems in the art by utilizing a novel biologically motivated evaluation framework (e.g., in a structured hierarchy) of a plurality of transcriptomic evaluation metrics to assess transcriptomics machine learning models (or foundation models) in a clear and systematic way. Further, the disclosed systems can introduce a novel structural integrity metric (e.g., as part of the plurality of transcriptomic evaluation metrics) for assessing gene activity structure preservation.

In some embodiments, the disclosed systems can utilize a transcriptomics machine learning model to generate embeddings from observed (or actual) gene expression profiles resulting from perturbations. Based on the embeddings, the disclosed systems can generate an evaluation framework of transcriptomic evaluation metrics, which includes the structural integrity metric. For instance, to generate the structural integrity metric, the disclosed systems may reconstruct predicted transcriptomic profiles from the embeddings. The disclosed systems can also generate adjusted transcriptomic profiles by applying control profiles to corresponding transcriptomic profiles. In at least one embodiment, to generate the structural integrity metric, the disclosed systems determine a structural distance between adjusted observed transcriptomic profiles and adjusted predicted transcriptomic profiles and compare the structural distance to a determined threshold (e.g., maximum) structural distance. Upon generating the structural integrity metric and other transcriptomic evaluation metrics within the evaluation framework, the disclosed systems can combine the transcriptomic evaluation metrics to generate a transcriptomic benchmark that extensively evaluates perturbation-related evaluation tasks across a transcriptomics modality.

### BRIEF DESCRIPTION OF THE DRAWINGS

The detailed description provides one or more embodiments with additional specificity and detail through the use of the accompanying drawings, as briefly described below.

FIG. 1 illustrates an example overview of generating a transcriptomic benchmark for a transcriptomics machine learning model in accordance with one or more embodiments.

FIG. 2 illustrates an example diagram of utilizing a transcriptomics machine learning model to generate transcriptomic embeddings from observed transcriptomic profiles of cells exposed to perturbations in accordance with one or more embodiments.

FIGS. 3A-3B illustrate an example diagram of generating a plurality of transcriptomic evaluation metrics in accordance with one or more embodiments.

FIG. 4 illustrates an example diagram of generating a structural integrity metric by generating a structural distance for a transcriptomics machine learning model in accordance with one or more embodiments.

FIG. 5 illustrates an example diagram of generating a structural distance by comparing adjusted observed transcriptomic profiles and adjusted predicted transcriptomic profiles in accordance with one or more embodiments.

FIG. 6 illustrates an example diagram of generating a structural integrity metric by comparing a structural distance to a threshold structural distance in accordance with one or more embodiments.

FIG. 7 illustrates an example diagram of training a transcriptomics machine learning model based on machine learning data associated with a structural integrity metric and, optionally, one or more other transcriptomic metrics in accordance with one or more embodiments.

FIG. 8 illustrates an example table that summarizes a performance comparison of various transcriptomics machine learning models on various evaluation tasks and metrics using transcriptomic benchmark datasets in accordance with one or more embodiments.

FIG. 9 illustrates a diagram of an example environment in which a transcriptomics model benchmarking system can operate in accordance with one or more embodiments.

FIG. 10 illustrates an example flowchart of a series of acts for generating a transcriptomic benchmark in accordance with one or more embodiments.

FIG. 11 illustrates a block diagram of an exemplary computing device in accordance with one or more embodiments.

### DETAILED DESCRIPTION

This disclosure describes one or more embodiments of a transcriptomics model benchmarking system 100 that can uniquely analyze transcriptomic machine learning models utilizing an evaluation framework of a plurality of transcriptomic evaluation metrics, which includes a structural integrity metric, to generate a transcriptomic benchmark that extensively evaluates perturbation-related evaluation tasks across a transcriptomics modality. In some embodiments, the transcriptomics model benchmarking system 100 can utilize a transcriptomics machine learning model to generate transcriptomic embeddings from observed (or actual) transcriptomic profiles resulting from perturbations. The transcriptomics model benchmarking system 100 can utilize the transcriptomic embeddings to generate the evaluation framework of the plurality of transcriptomic metrics, which includes the structural integrity metric. For instance, to generate the structural integrity metric, the transcriptomics model benchmarking system 100 may reconstruct predicted transcriptomic profiles from the transcriptomic embeddings. The transcriptomics model benchmarking system 100 can also generate adjusted transcriptomic profiles by applying corresponding control profiles to the transcriptomic profiles. In at least one embodiment, to generate the structural integrity metric, the transcriptomics model benchmarking system 100 determines a structural distance between adjusted observed transcriptomic profiles and adjusted predicted transcriptomic profiles and compares the structural distance to a determined threshold (e.g., maximum) structural distance. Further, the transcriptomics model benchmarking system 100 can generate the transcriptomic benchmark by combining the generated structural integrity metric with other generated transcriptomic evaluation metrics.

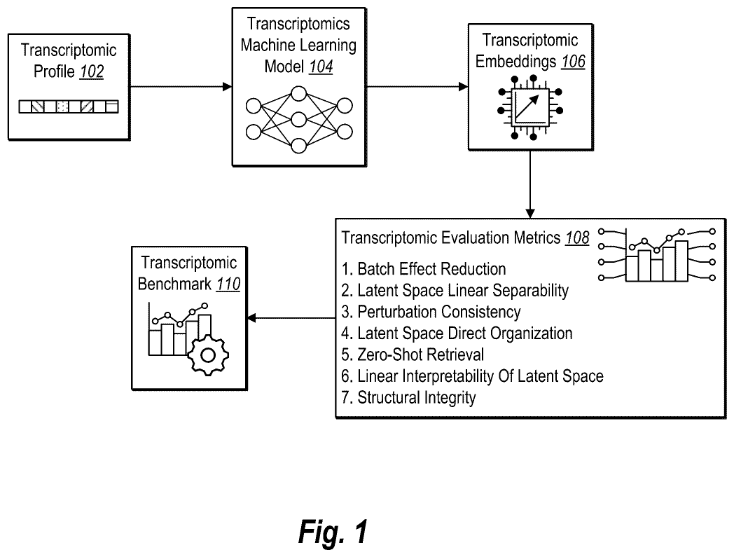

For example, FIG. 1 illustrates an example overview of generating a transcriptomic benchmark for a transcriptomics machine learning model in accordance with one or more embodiments. Additional detail regarding the various acts and processes introduced in relation to FIG. 1 is provided thereafter with reference to subsequent figures.

As illustrated in FIG. 1, the transcriptomics model benchmarking system 100 utilizes data from a transcriptomic profile 102 of a gene (e.g., a gene expression profile) resulting from perturbations to generate transcriptomic embeddings 106 of the transcriptomic profile 102. In particular, the transcriptomics model benchmarking system 100 can utilize a transcriptomics machine learning model 104 (e.g., a transcriptomics foundation model) to generate the transcriptomic embeddings 106. For instance, the transcriptomics model benchmarking system 100 can utilize data from one or more transcriptomic profiles (e.g., from one or more observed transcriptomic profiles) resulting from perturbations as machine learning data for the transcriptomics machine learning model 104. Based on the machine learning data of the one or more transcriptomic profiles, the transcriptomics model benchmarking system 100 can generate the transcriptomic embeddings 106 within a low-dimensional, continuous vector space.

As also illustrated in FIG. 1, the transcriptomics model benchmarking system 100 can generate a plurality of transcriptomic evaluation metrics 108. In particular, the transcriptomics model benchmarking system 100 can utilize the transcriptomic embeddings 106 to generate the plurality of transcriptomic evaluation metrics 108. In some embodiments, the transcriptomics model benchmarking system 100 can generate an evaluation framework of the plurality of transcriptomic evaluation metrics 108. Specifically, the transcriptomics model benchmarking system 100 can generate the evaluation framework to assess a machine learning model (e.g., the transcriptomics machine learning model 104 and, optionally, one or more other machine learning models) in a clear and systematic way. In some embodiments, the transcriptomics model benchmarking system 100 generates, as part of the evaluation framework, a structured hierarchy of the plurality of transcriptomic evaluation metrics 108. In some cases, when assessing the machine learning model, the transcriptomics model benchmarking system 100 can utilize the structured hierarchy to progress through the plurality of transcriptomic evaluation metrics 108 in a predetermined order and/or utilizing predetermined weights.

As further illustrated in FIG. 1, the transcriptomics model benchmarking system 100 generates the plurality of transcriptomic evaluation metrics 108 to assess the performance and effectiveness of the transcriptomics machine learning model 104 in specific tasks. The transcriptomics model benchmarking system 100 can generate the plurality of transcriptomic evaluation metrics 108 to assess, for example: (1) data integrating and batch effect reduction; (2) latent space linear separability of known perturbations; (3) perturbation consistency; (4) latent space direct organization; (5) zero-shot retrieval of known biological relationships; (6) linear interpretability of the latent space; and/or (7) structural integrity (or structure preservation) of gene activity. In at least one embodiment, when assessing the machine learning model, the transcriptomics model benchmarking system 100 utilizes the structured hierarchy to progress through the plurality of transcriptomic evaluation metrics 108 in the order just described. Similarly, in some implementations, the transcriptomics model benchmarking system 100 can apply weights in a hierarchical order/emphasis based on the foregoing order (or a different order).

In the same or other embodiments, the transcriptomics model benchmarking system 100 generates a structural integrity metric to assess how well the transcriptomics machine learning model 104 preserves gene activity structure (e.g., the structural integrity of gene activity). In particular, the transcriptomics model benchmarking system 100 may use a neural network (e.g., a multilayer perceptron or “MLP”) to reconstruct predicted transcriptomic profiles from the transcriptomic embeddings 106. In some instances, the transcriptomics model benchmarking system 100 generates adjusted observed transcriptomic profiles and adjusted predicted transcriptomic profiles by applying corresponding control profiles to observed transcriptomic profiles and the predicted transcriptomic profiles. In at least one embodiment, the transcriptomics model benchmarking system 100 determines a structural distance between adjusted observed transcriptomic profiles and adjusted predicted transcriptomic profiles. Upon determining the structural distance, the transcriptomics model benchmarking system 100 may complete generating the structural integrity metric by comparing the structural distance to a determined threshold (e.g., maximum) structural distance.

As shown in FIG. 1, the transcriptomics model benchmarking system 100 can generate a transcriptomic benchmark 110 to evaluate a performance of the transcriptomics machine learning model 104. In the same or other embodiments, the transcriptomics model benchmarking system 100 generates the transcriptomic benchmark 110 to evaluate, compare, and/or validate the performance of one or more transcriptomics machine learning models (and/or transcriptomic evaluation methods, such as baseline approaches like PCA and scVI) in analyzing transcriptomic data (e.g., perturbations). In particular, the transcriptomics model benchmarking system 100 can generate the transcriptomic benchmark 110 by utilizing the plurality of transcriptomic evaluation metrics 108. For instance, upon generating the plurality of transcriptomic evaluation metrics 108, which includes the structural integrity metric, the transcriptomics model benchmarking system 100 generates the transcriptomic benchmark 110 by combining the plurality of transcriptomic evaluation metrics 108. As an example, the transcriptomic benchmark 110 includes a combined benchmark score and/or a table, graph, chart, and/or other dataset.

As mentioned above, conventional systems exhibit a number of technical deficiencies or drawbacks. For instance, conventional systems lack functionality. As an example, while some conventional systems use machine learning models to generate transcriptomics embeddings, conventional systems lack an approach for robustly evaluating the effectiveness of these models for perturbation analysis. To elaborate, although conventional systems often focus on tasks like cell type classification and clustering, benchmarks for evaluating transcriptomic machine learning models in perturbation analysis remain limited. Indeed, rather than generating a comprehensive benchmark that extensively evaluates perturbation-related evaluation tasks for the transcriptomics modality, conventional systems often generate limited benchmarks based on evaluating a particular aspect of a particular model using a specific metric. As a result of such limited benchmarking, conventional systems are unable to clearly determine which machine learning models are most effective for perturbation analysis.

Due at least in part to the lack of functionality, existing systems are also inaccurate. For instance, as just mentioned, existing systems often only evaluate a particular aspect of a particular machine learning model using a specific metric. However, by so doing, such systems only obtain a fragmentary, partial, and/or inaccurate evaluation. As a result, existing systems often fail to identify which machine learning models deliver the most effective and holistic perturbation analyses. Indeed, this narrow focus on an isolated task or a particular aspect of a model results in evaluations based solely on a partial performance metric, which fails to account for a model's overall ability to capture the complexity of cellular changes or results from applied perturbations.

Furthermore, existing systems are also inaccurate because they are often only trained on narrowly defined datasets, tasks, and/or metrics. In particular, due to being trained on narrowly defined datasets, tasks, and/or metrics, existing systems may overfit and capture dataset-specific noise rather than biologically relevant signals. This can lead to inaccurate generalization when applied to new perturbations or experimental conditions. Moreover, existing systems' often produce biased or misleading conclusions regarding model effectiveness.

In addition to their functionality and accuracy shortcomings, prior systems are also inefficient. For example, because prior systems are often only trained on narrowly defined datasets, tasks, and/or metrics, prior systems are inefficient in their use of training loss functions. As a result, prior systems require more computational effort and time to adjust their internal parameters effectively, leading to an inefficient process for the model to improve its performance and reach a point where it makes accurate predictions (e.g., converges).

In one or more embodiments, the transcriptomics model benchmarking system 100 can provide several technological improvements or advantages relative to existing systems. For instance, the transcriptomics model benchmarking system 100 can improve functionality over prior systems. While conventional systems may generate limited benchmarks based on evaluating a particular aspect of a particular machine learning model, the transcriptomics model benchmarking system 100 can generate a more comprehensive transcriptomic benchmark that extensively evaluates perturbation-related evaluation tasks across a transcriptomics modality. To elaborate, in at least one embodiment, the transcriptomics model benchmarking system 100 uses a biologically motivated benchmarking tool for evaluating one or more transcriptomics machine learning models on a plurality of perturbation relevant tasks. Indeed, the transcriptomics model benchmarking system 100 can utilize a unique evaluation framework (e.g., in a structured hierarchy) of a plurality of transcriptomic evaluation metrics, which includes a novel structural integrity metric, to generate the more comprehensive transcriptomic benchmark for each of the one or more transcriptomic machine learning models. Using the compressive transcriptomic benchmark for each of the one or more transcriptomic machine learning models, the transcriptomics model benchmarking system 100 can determine which model(s) is (are) most effective for perturbation analysis. For instance, the transcriptomics model benchmarking system 100 intelligently compares the performance of models to other models and/or to transcriptomic evaluation methods (e.g., methods or techniques of learning from transcriptomics data) to determine which model(s) and/or method(s) is (are) most effective for perturbation analysis.

Not only does utilizing the unique evaluation framework of the plurality of transcriptomic evaluation metrics to generate a more comprehensive transcriptomic benchmark provide for greater functionality but it also improves accuracy relative to existing systems. For instance, in contrast with existing systems that evaluate machine learning models based on a partial performance metric, the transcriptomics model benchmarking system 100 can evaluate a transcriptomics machine learning model based on the evaluation framework of the plurality of transcriptomic evaluation metrics that includes the structural integrity metric. In some embodiments, the transcriptomics model benchmarking system 100 can utilize a structured hierarchy of transcriptomic evaluation metrics to assess the models in a clear and systematic way.

Furthermore, in particular embodiments, the transcriptomics model benchmarking system 100 generates and/or utilizes, as part of the evaluation framework, the structural integrity metric to assess how well model embeddings preserve the relationship between control and perturbation conditions within each biological batch in the gene activity dimension. Indeed, the structural integrity metric can be crucial for utilizing reconstructed transcriptomic profiles to accurately study gene expression changes under different conditions. For instance, by adjusting both observed transcriptomic profiles and predicted transcriptomic profiles relative to control transcriptomic profiles, the structural integrity metric generates measurements that reflect changes caused by perturbations relative to control conditions within each biological batch (rather than just capturing noise or variability unrelated to a biological effect of interest).

Moreover, the transcriptomics model benchmarking system 100 can also improve the accuracy of machine learning models (e.g., transcriptomics machine learning models) relative to existing systems. In contrast with existing systems, the transcriptomics model benchmarking system 100 can modify the parameters of (e.g., train) one or more models based on machine learning data associated with the evaluation framework (e.g., as the structured hierarchy) of the plurality of transcriptomic evaluation metrics. For instance, upon generating the structural integrity metric and/or the other transcriptomic evaluation metrics of the evaluation framework, the transcriptomics model benchmarking system 100 can train one or more models on machine learning data associated with the structural integrity metric and, optionally, one or more of the other transcriptomic evaluation metrics. By training the one or more models in at least one of these ways, the transcriptomics model benchmarking system 100 can enable the one or more models to more accurately capture biologically relevant signals (rather than dataset-specific noise or other inaccurate generalizations often captured by existing systems). In addition, the transcriptomics model benchmarking system 100 can also improve efficiency. For instance, the transcriptomics model benchmarking system 100 can more efficiently train a machine learning model (e.g., to converge more quickly requiring less computational resources and time).

As illustrated by the foregoing discussion, the present disclosure utilizes a variety of terms to describe features and advantages of the video transcript segmentation system. Additional detail is now provided regarding the meaning of such terms. As used herein, the term “transcriptomic” refers to an examination of gene activity at the RNA level within a biological system. In particular, the term transcriptomic can refer to the study and analysis of a transcriptome, which is a set of RNA transcripts produced by the genome of a cell, tissue, and/or organism at a specific time or under particular conditions. In some embodiments, the transcriptome includes various RNA species such as messenger RNA (mRNA), ribosomal RNA (rRNA), transfer RNA (RNA), and/or non-coding RNAs (ncRNA). In the same or other embodiments, the term transcriptomic encompasses methods, techniques, and/or computational approaches used to quantify, analyze, and/or interpret the expression, regulation, and functional roles of these RNA molecules. Transcriptomic studies may rely on high-throughput technologies like RNA sequencing (RNA-Seq) or microarrays to generate large-scale data.

Relatedly, as used herein, the term “transcriptomic profile” refers to a quantitative representation of gene expression activity within the transcriptome of a biological sample (e.g., a cell). In particular, a transcriptomic profile can serve as a snapshot of the functional state of the transcriptome, encompassing a set (e.g., the entire set) of RNA molecules expressed from the genome (e.g., RNA transcripts, such as messenger RNA, produced during gene transcription). In some cases, a transcriptomic profile is a matrix, array, or vector where each dimension corresponds to an expression level of a particular gene (for a particular perturbation).

Along those lines, as used herein, the term “observed transcriptomic profile” (or “actual transcriptomic profile) refers to an experimentally derived transcriptomic profile. For instance, an observed transcriptomic profile reflects the state of RNA abundance within a sample, in some cases capturing both baseline and dynamically regulated transcripts. Indeed, an observed transcriptomic profile can provide a high-dimensional dataset that represents the transcriptomic landscape as observed in the experimental data. Relatedly, as used herein, the term “adjusted observed transcriptomic profile” refers to an observed transcriptomic profile that has been modified (or adjusted) with (or relative to) a control transcriptomic profile.

In addition, as used herein, the term “predicted transcriptomic profile” refers to a computationally inferred representation of a transcriptomic profile. In particular, a predicted transcriptomic profile can be generated using a machine learning model, such as a neural network. To elaborate, a predicted transcriptomic profile may be derived based on input data (e.g., prior experimental profiles, genomic features, environmental conditions, and/or perturbation simulations) rather than direct experimental measurements. As an example, the transcriptomics model benchmarking system 100 may use a neural network (e.g., a multilayer perceptron or “MLP”) to generate predicted transcriptomic profiles by reconstructing transcriptomic profiles from transcriptomic embeddings. Relatedly, as used herein, the term “adjusted predicted transcriptomic profile” refers to a predicted transcriptomic profile that has been modified (or adjusted) with (or relative to) a control transcriptomic profile.

Moreover, as used herein, the term “control transcriptomic profile” refers to a transcriptomic dataset that represents the baseline or reference gene expression levels in a biological sample under standard, untreated, and/or unperturbed conditions (e.g., unperturbed samples in the same batch). In particular, a control transcriptomic profile can serve as a benchmark for comparison in experimental studies (e.g., to identify changes in gene expression resulting from perturbations). As an example, a control profile may be derived from samples that are maintained under well-defined, consistent conditions to minimize variability so that observed differences in observed transcriptomic profiles are more attributable to the variable being tested.

As also used herein, the term “batch” refers to a set of samples processed in a common group (e.g., different batches indicate samples processed at different times or conditions). In particular, a batch can, in some cases, refer to a set or collection of biological and/or experimental samples that are processed together under similar conditions and analyzed as a group. Similarly, as used herein, the term “sample” refers to a biological entity or unit from which transcriptomic data is collected. For instance, a sample may represent an instance in a dataset and can be characterized by a transcriptomic profile. As an example, a perturbed cell can be considered a sample.

As further used herein, the term “perturbation” refers to an alteration or disruption to a biological system, such as a cell or the cell's environment. In some cases, perturbations are used, for example, to elicit potential phenotypic changes to a biological system and/or to observe how a biological system responds at the transcriptomic level. Example perturbations include, but are not limited to, genetic interventions (e.g., CRISPR-based gene editing; RNA interference (RNAi); or mutagenesis), chemical perturbations (e.g., exposing cells to drugs or other chemicals, small molecules, or hormones), environmental perturbations (e.g., stress conditions, such as temperature changes, nutrient deprivation, hypoxia, or exposure to a physical force like mechanical stress or electrocution), and infection or co-culture exposure (e.g., exposure to a pathogen). Further, the term perturbation can include a small molecule perturbation (e.g., a compound perturbation), a protein perturbation, an antibody perturbation, a gene perturbation, a virus perturbation, or an in vivo perturbation.

As used herein, the term “machine learning model” refers to a computer algorithm or a collection of computer algorithms that improve for a particular task through iterative outputs or predictions based on use of data. For example, a machine learning model can utilize one or more learning techniques to improve in accuracy and/or effectiveness. Example machine learning models include various types of decision trees, support vector machines, Bayesian networks, random forest models, or neural networks (e.g., deep neural networks, generative adversarial neural networks, convolutional neural networks, recurrent neural networks, or diffusion neural networks). Similarly, the term “machine learning data” refers to information, data, or files generated or utilized by a machine learning model. Machine learning data can include training data, machine learning parameters, or embeddings/predictions generated by a machine learning model.

As additionally used herein, the term “neural network” refers to a machine learning model that can be trained and/or tuned based on inputs to approximate unknown functions. For example, a neural network includes a model of interconnected artificial neurons (e.g., organized in layers) that communicate and learn to approximate complex functions and generate outputs based on a plurality of inputs provided to the neural network. In some cases, a neural network refers to an algorithm (or a set of algorithms) that implements deep learning techniques to model high-level abstractions in data. A neural network can include various layers such as an input layer, one or more hidden layers, and an output layer that each perform tasks for processing data. For example, a neural network can include a deep neural network a convolutional neural network, a recurrent neural network (e.g., an LSTM), a graph neural network, a large language model, or a generative neural network.

Relatedly, as used herein, the term “transcriptomics machine learning model” (e.g., a transcriptomics foundation model) refers to machine learning model trained to analyze transcriptomic data and/or to predict changes in gene expression resulting from perturbations. In some cases, a transcriptomics machine learning model can leverage transcriptomic datasets and machine learning techniques.

Additionally, as used herein, the term “transcriptomic benchmark” refers to a metric, measure, and/or dataset of perturbation data. In particular, a transcriptomic benchmark can be used to evaluate, compare, and/or validate the performance of one or more transcriptomics machine learning models and/or one or more transcriptomic evaluation methods (e.g., methods or techniques of learning from transcriptomics data, such as PCA and scVI) in analyzing transcriptomic data (e.g., perturbations). For instance, a transcriptomic benchmark can provide a reference framework for assessing the ability of a transcriptomics machine learning model to accurately capture and predict changes in gene expression resulting from specific biological perturbations.

Further, as used herein, the term “transcriptomic embedding” refers to vector representation (or embedding) of high-dimensional transcriptomic data. For instance, the transcriptomics model benchmarking system 100 generates transcriptomic embeddings within a low-dimensional, continuous vector space based on machine learning data from the one or more transcriptomic profiles.

As used herein, the term “transcriptomic evaluation metric” refers to a quantitative measure used to assess the performance and effectiveness of machine learning models in a specific task. In one or more embodiments, transcriptomic evaluation metrics provide an evaluation framework for comparing transcriptomics machine learning models by evaluating how well each performs in areas such as, for example, data integration and batch effect reduction, latent space linear separability of known perturbations, perturbation consistency, latent space direct organization, zero-shot retrieval of known biological relationships, a linear interpretability of latent space (e.g., using Spearman correlation), and/or gene activity structural integrity (or structure preservation).

Along those lines, as used herein, the term “structural integrity metric” refers to a transcriptomic evaluation metric utilized by the transcriptomics model benchmarking system 100 to assess how well a transcriptomics machine learning model preserves gene activity structure. In particular, the structural integrity metric is a novel transcriptomic evaluation metric that can assess how well a transcriptomics machine learning model's embeddings preserve the relationship between control and perturbation conditions with each biological batch in a gene activity dimension. In one or more embodiments, higher values of structural integrity indicate better preservation of structural relationships in gene expression data for a transcriptomics machine learning model.

Additionally, as used herein, the term “structural distance” refers to a quantitative measure used within the structural integrity metric to evaluate how well a model preserves the relationship between control and perturbation conditions in gene expression profiles, while accounting for batch-specific variability. Specifically, the structural distance can quantify (e.g., in an original gene expression space) the difference between observed transcriptomic profiles and predicted transcriptomic profiles that are each adjusted relative to control samples for each biological batch.

Relatedly, as used herein, the term “threshold structural distance” (or maximum structural distance) refers to a bound (e.g., theoretically upper bound) for the structural distance. In particular, the threshold structural distance can refer to the highest possible value that the structural distance can take, given the size and characteristics of the gene expression data. Specifically, the threshold structural distance can represent the maximum difference that could theoretically exist between adjusted observed transcriptomic profiles and adjusted predicted transcriptomic profiles across batches. In some cases, the threshold structural distance is used to normalize the structural distance when calculating the structural integrity metric, providing a scale-free measure of performance.

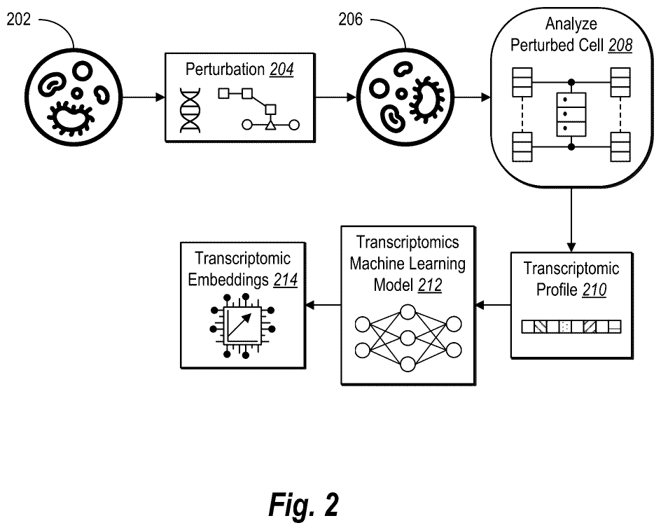

Additional detail regarding the transcriptomics model benchmarking system 100 will again be provided with reference to the figures. For example, as mentioned above, the transcriptomics model benchmarking system 100 can generate transcriptomic embeddings of a transcriptomic profile. In particular, the transcriptomics model benchmarking system 100 can utilize a transcriptomics machine learning model to generate the transcriptomic embeddings. FIG. 2 illustrates an example diagram of utilizing a transcriptomics machine learning model to generate transcriptomic embeddings from observed transcriptomic profiles of cells exposed to perturbations in accordance with one or more embodiments.

As illustrated in FIG. 2, the transcriptomics model benchmarking system 100 can determine that a cell 202 has undergone one or more perturbations (e.g., perturbation 204), resulting in a perturbed cell 206 sample. To elaborate, the cell 202 may be a single cell (e.g., a cell isolated by transcriptomic profiling, such as in single-cell RNA sequencing), a cell line (e.g., a homogeneous population of cells cultured in vitro), a primary cell (e.g., a cell taken directly from living tissues), a differentiated cell (e.g., a cell that has been induced to develop into specific cell types, such as a neuron), and/or a cell collective (e.g., a group of cells pooled together). In some embodiments, the cell 202 can be an unperturbed cell. In the same or other embodiments, the cell 202 may be a perturbed cell that has previously undergone a perturbation. Upon undergoing one or more perturbations, the resulting perturbed cell 206 may exhibit an alteration or disruption (e.g., a modified gene expression, behavior, or molecular state) that may not have been present in the cell 202.

As also illustrated in FIG. 2, the transcriptomics model benchmarking system 100 performs the act 208 to analyze the perturbed cell 206. In particular, upon determining that the cell 202 has undergone one or more perturbations, the transcriptomics model benchmarking system 100 can identify the perturbed cell 206. Based on identifying the perturbed cell 206, the transcriptomics model benchmarking system 100 can utilize computer hardware (e.g., a transcriptomics machine) to perform the act 208. In some embodiments, the transcriptomics model benchmarking system 100 may extract RNA from the perturbed cell 206 and/or perform RNA sequencing (or single-cell sequencing) on the perturbed cell 206 prior to (or as a part of) performing the act 208.

In the same or other embodiments, the transcriptomics model benchmarking system 100 performs the act 208 by processing raw reads (e.g., raw sequencing data) into a usable format that accurately reflects gene expression levels. In particular, the transcriptomics model benchmarking system 100 can process the raw reads obtained from performing RNA sequencing (or single-cell sequencing) on the perturbed cell 206. Processing the raw sequencing data may be done using a series of steps that may include (but is not limited to): quality-checking the raw reads; aligning the raw reads to a reference genome or transcriptome using alignment tools (e.g., HISAT2 or STAR) to map each raw read to specific genes or exonic regions; quantifying the number of the raw reads corresponding to each gene (e.g., to quantify gene expression levels); and/or normalizing the data (e.g., to ensure comparability across samples).

As further illustrated in FIG. 2, the transcriptomics model benchmarking system 100 generates a transcriptomic profile 210 for the perturbed cell 206. In particular, the transcriptomics model benchmarking system 100 can generate the transcriptomic profile 210 by creating a quantitative representation of gene expression for the perturbed cell 206. For instance, upon processing the raw reads, the transcriptomics model benchmarking system 100 can organize the processed raw reads data to generate the transcriptomic profile 210. As an example, the transcriptomics model benchmarking system 100 may organize the processed raw reads data into a structured format (e.g., an array, matrix, or vector) that quantitatively represents the expression levels of genes for the perturbed cell 206 sample.

As shown in FIG. 2, the transcriptomics model benchmarking system 100 utilizes a transcriptomics machine learning model 212 (e.g., a transcriptomics foundation model) to generate transcriptomic embeddings 214 of the transcriptomic profile 210. In particular, the transcriptomics model benchmarking system 100 may input data from the transcriptomic profile 210 into the transcriptomics machine learning model 212. Based on this input, the transcriptomics model benchmarking system 100 may utilize the transcriptomics machine learning model 212 to transform the transcriptomic profile 210 data (e.g., a high-dimensional vector representation) into the transcriptomic embedding 214 (e.g., a low-dimensional, continuous vector representation). The transcriptomics model benchmarking system 100 can utilize a variety of different machine learning models or architectures for the transcriptomics machine learning model 202, including, but not limited to, sc VI, Geneformer, scGPT, CellPLM, Universal Cell Embeddings or “UCE,” scBERT, and/or scVAEIT. The transcriptomics model benchmarking system 100 can also utilize a masked autoencoder, as described in UTILIZING MASKED AUTOENCODER GENERATIVE MODELS TO EXTRACT MICROSCOPY REPRESENTATION AUTOENCODER EMBEDDINGS, U.S. patent application Ser. No. 18/545,399, filed Dec. 19, 2023, which is incorporated herein by reference in its entirety.

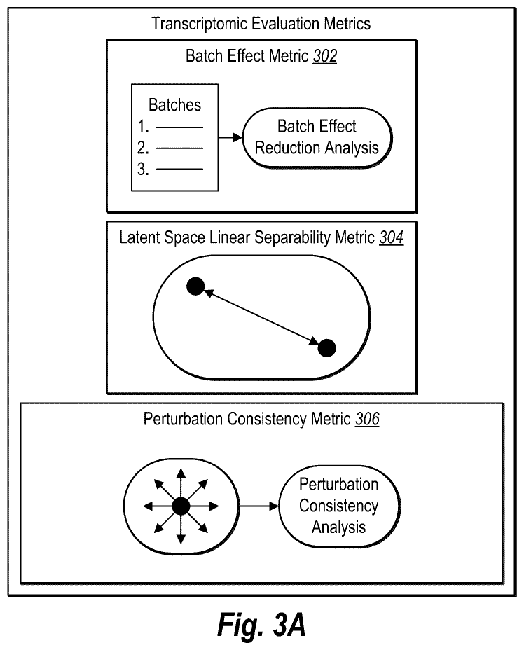

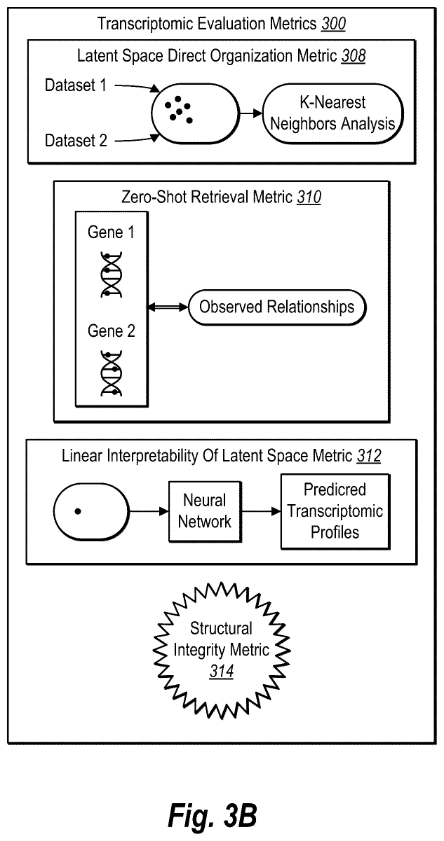

As expressed above, in some embodiments, the transcriptomics model benchmarking system 100 can generate a plurality of transcriptomic evaluation metrics. In particular, the transcriptomics model benchmarking system 100 can utilize transcriptomic embeddings to generate the plurality of transcriptomic evaluation metrics. FIGS. 3A-3B illustrate an example diagram of generating a plurality of transcriptomic evaluation metrics in accordance with one or more embodiments.

As shown in FIGS. 3A-3B, the transcriptomics model benchmarking system 100 generates one or more transcriptomic evaluation metrics 300 to assess the performance and effectiveness of a transcriptomics machine learning model in specific tasks. For instance, the transcriptomics model benchmarking system 100 can generate a plurality of transcriptomic evaluation metrics. For example, the transcriptomics model benchmarking system 100 generates one or more of: a batch effect metric 302 to assess data integrating and batch effect reduction; a latent space linear separability metric 304 to assess latent space linear separability of known perturbations; a perturbation consistency metric 306 to assess perturbation consistency; a latent space direct organization metric 308 to assess latent space direct organization; a zero-shot retrieval metric 310 to assess zero-shot retrieval of known biological relationships; a linear interpretability of latent space metric 312 to assess linear interpretability of the latent space; and/or a structural integrity metric 314 to assess structural integrity (or structure preservation) of gene activity. In the same or other embodiments, the one or more transcriptomic evaluation metrics 300 can include one or more other metrics in addition to the one or more metrics illustrated in FIGS. 3A-3B.

As illustrated in FIG. 3A, and as just mentioned, the transcriptomics model benchmarking system 100 can generate the batch effect metric 302. To elaborate, in biological experiments, data often comes from different batches. In some cases, batch differences can introduce artificial variations known as “batch effects,” which can obscure true perturbation or treatment response signals. For perturbation analysis, it can be important for a transcriptomics machine learning model to integrate data from multiple batches seamlessly to ensure that comparisons between samples reflect real biological differences, not technical artifacts.

In some implementations, the transcriptomics model benchmarking system 100 can implement the batch effect metric 302 as an Integration Local Inverse Simpson's Index (iLISI) metric. In particular, the iLISI metric can measure how well a transcriptomics machine learning model reduces batch effects. Indeed, the iLISI metric may assess how mixed samples from different batches are within the transcriptomics machine learning model's representation space. For instance, if, in the vicinity (e.g., neighborhood) of any given sample, there is a good mix of samples from all batches, this may suggest that the transcriptomics machine learning model has effectively minimized batch effects.

To calculate the iLISI score, the transcriptomics model benchmarking system 100 may identify the closest neighboring samples for each data point based on their similarity or distance. The transcriptomics model benchmarking system 100 may then assign a probability to each neighbor, where closer neighbors are given higher probabilities, reflecting their stronger similarity to the sample in question. To ensure consistency across the dataset, the transcriptomics model benchmarking system 100 may then apply a scaling factor so that the number of neighbors considered for each sample matches a predetermined target. This adjustment can improve uniformity and allow for meaningful comparisons. In some embodiments, the resulting iLISI score provides an indication of how effectively samples from different batches are integrated, highlighting whether batch-specific artifacts have been minimized in the data.

In one or more embodiments, to compute the iLISI score, the transcriptomics model benchmarking system 100 defines the conditional probability pᵢⱼ of sample i selecting sample j as a neighbor, with dᵢⱼ being the distance between samples i and j, βᵢ being a scaling parameter adjusted such that the entropy H(Pᵢ)=log(k), ensuring the number of nearest neighbors matches the target, and Nᵢ is the set of k nearest neighbors of sample i:

$${p}_{ij} = \frac{exp ( - {β}_{i} {d}_{ij} )}{{\sum }_{l ∈ {𝒩}_{i}} exp ( - {β}_{i} {d}_{il} )}$$

In some embodiments, with n being the total number of samples, C the set of all possible categories (batch labels), and lⱼ the label (e.g., batch category) of neighbor j, the iLISI score is then calculated as:

$$iLISI = \frac{1}{n} {\sum }_{i = 1}^{n} {( {\sum }_{c ∈ C} {( {{\sum }_{j ∈ {𝒩}_{i}}}_{{l}_{j} = c} )}^{2} )}^{- 1}$$

As also shown in in FIG. 3A, and as mentioned above, the transcriptomics model benchmarking system 100 can generate the latent space linear separability metric 304. In particular, the transcriptomics model benchmarking system 100 can generate the latent space linear separability metric 304 to assess how the transcriptomics machine learning model distinguishes between different perturbations. For example, the transcriptomics machine learning model's internal “map” (or latent space) can be assessed to see how well it reflects the biological differences caused by various perturbations. The capacity of the transcriptomics machine learning model to do this can be important for identifying how different perturbations (or interventions) affect biological systems (e.g., cells).

In at least one embodiment, to assess the latent space linear separability of known perturbations, the transcriptomics model benchmarking system 100 determines whether one or more samples subjected to different perturbations can be separated using a linear classifier for linear probing (e.g., to determine how well the learned features in the transcriptomics machine learning model's latent space capture the information needed to distinguish between different types of perturbations). Specifically, the transcriptomics model benchmarking system 100 can add a linear layer on top of representations generated utilizing the transcriptomics machine learning model to classify the one or more samples based on their perturbations. In some cases, the transcriptomics model benchmarking system 100 evaluates classifier performance on the same perturbations but on new biological batches of data that the transcriptomics machine learning model has not seen before. This can help test whether the transcriptomics machine learning model is memorizing noise patterns of the training data or able to generalize to new, unseen data.

As further illustrated in FIG. 3A, and as mentioned above, the transcriptomics model benchmarking system 100 can generate the perturbation consistency metric 306. In particular, the transcriptomics model benchmarking system 100 can generate the perturbation consistency metric 306 to assess how well the transcriptomics machine learning model consistently represents each perturbation across various samples and batches. To elaborate, such consistency can help test if the transcriptomics machine learning model is robust and whether representations are reliable and not influenced by noise or outliers.

In some implementations, the transcriptomics model benchmarking system 100 generates the perturbation consistency metric 306 to assess how consistently a perturbation is represented across different samples and batches. Specifically, the transcriptomics model benchmarking system 100 can calculate the similarity between the embeddings of all sample pairs for that perturbation. Specifically, for each perturbation g, the transcriptomics model benchmarking system 100 can calculate cosine similarity between pairs of the perturbation's embeddings from different samples and batches (e.g., to measure how closely the embeddings align). The average of these cosine similarity scores across sample pairs can give a perturbation similarity score for the perturbation.

Formally, in at least one embodiment, the transcriptomics model benchmarking system 100 calculates a per-perturbation similarity score as follows: let x_{g,i} be the embedding vector for the i-th sample of perturbation g, and n_{g} be the number of samples for g. The per-perturbation similarity score avgsim_{g} can then be computed as:

$${avgsim}_{g} = \frac{1}{{n}_{g}^{2}} {\sum }_{i = 1}^{{n}_{g}} {\sum }_{j = 1}^{{n}_{g}} \frac{〈 {x}_{g , i} , {x}_{g , j} 〉}{‖ {x}_{g , i} ‖ ‖ {x}_{g , j} ‖}$$

In the same or other embodiments, upon performing the above computation, the transcriptomics model benchmarking system 100 can compare the per-perturbation similarity score to a null distribution generated from unexpressed genes (e.g., genes that are inactive in the dataset). The transcriptomics model benchmarking system 100 can select unexpressed genes based, for instance, on their consistently low expression levels. In some cases, the transcriptomics model benchmarking system 100 selects at least 1,000 unexpressed genes to obtain more meaningful results.

In at least one embodiment, for each unexpressed gene g′ₖ, k=1, . . . , K, the transcriptomics model benchmarking system 100 computes their average cosine similarity avgsim_{g′}_{ₖ} in the same way. Using a permutation test, the transcriptomics model benchmarking system 100 can assess whether the observed similarity for perturbation g is significantly higher than what would occur by chance. The consistency p-value for each gene g is given by:

$${p}_{g} = \frac{max \{ \# ( {avgsim}_{{g}_{k}^{′}} \leq {avgsim}_{g} ) , 1 \}}{K}$$

In one or more embodiments, the transcriptomics model benchmarking system 100 determines that genes that achieve a consistency above a particular threshold (e.g., p-value <0.05) are significant. Based on this, the perturbation consistency metric 306 can report a fraction of these determined significant genes compared to all genes. A high perturbation consistency score may indicate that the transcriptomics machine learning model consistently recognizes the effect of a perturbation across different batches and experiments. Conversely, a low perturbation consistency score can suggest that the transcriptomics machine learning model may not fully capture the perturbation's effect, potentially classifying it correctly in other metrics simply because of outlier behavior or similarity to other cases.

As illustrated in FIG. 3B, and as mentioned above, the transcriptomics model benchmarking system 100 can generate the transcriptomic evaluation metric 308. In particular, the transcriptomics model benchmarking system 100 can generate the transcriptomic evaluation metric 308 to assess how well the transcriptomics machine learning model self-organizes a latent space without additional help or training. In some instances, self-organizing includes similar perturbations naturally clustering together and/or dissimilar (or distinct) perturbations naturally separating apart, even in new, unseen data. This can be important, for example, when using the transcriptomics machine learning model in practical, exploratory settings where it might encounter new types of data but cannot be continuously finetuned.

In at least one embodiment, to assess the latent space direct organization, the transcriptomics model benchmarking system 100 uses two datasets with the same types of perturbations: a query set (e.g., for reference) and a test set (e.g., for testing). These two datasets can come from different experimental batches to avoid overlap. For each sample in the test set, the transcriptomics model benchmarking system 100 can determine each sample's closest match(es) (e.g., neighbor(s)) in the query set based on the latent space organization. In some cases, the transcriptomics model benchmarking system 100 calculates the accuracy of the match(es) to see if samples in the test set are correctly grouped with the same perturbations from the query set. This can ensure the latent space is well-organized and can generalize to new data.

For example, the transcriptomics model benchmarking system 100 can, in some instances, assess the latent space direct organization by applying a k-Nearest neighbors (kNN) while using two different sets of data with the same perturbations, a query set and test set, but with no overlap of biological batches. For each sample in the test set, the transcriptomics model benchmarking system 100 can analyze a given sample's closest neighbors in the latent space of the query set. In some embodiments, the transcriptomics model benchmarking system 100 computes the kNN accuracy using the samples from the test batches that correspond to the same perturbation as their closest neighbor from the query set of batches.

As also illustrated in FIG. 3B, and as mentioned above, the transcriptomics model benchmarking system 100 can generate the zero-shot retrieval metric 310. In particular, the transcriptomics model benchmarking system 100 can generate the zero-shot retrieval metric 310 to assess how well the transcriptomics machine learning model is able to capture biological relationships between genes and to discover new genes, without being explicitly trained to find these connections. This, for example, can be beneficial for generating new insights and validating biological relevance of the transcriptomics machine learning model.

In at least one implementation, the zero-shot retrieval metric 306 can be a known relationships retrieval metric. In some cases, the known relationships retrieval metric assesses how well gene-to-gene distances in a latent space corresponding to a transcriptomics machine learning model can retrieve known relationships from curated gene/protein interaction databases (e.g., CORUM, HuMAP, Reactome, SIGNOR, and/or StringDB). Specifically, the known relationships retrieval metric can evaluate the ability of a transcriptomics machine learning model to discover relationships that exist but were not explicitly provided during training. Thus, the known relationships retrieval metric can, for example, highlight the potential of a transcriptomics machine learning model in exploratory settings where unknown relationships can be sought.

In one or more embodiments, to compute the known relationships retrieval metric, the transcriptomics model benchmarking system 100 calculates how similar the embeddings (representations) of perturbed genes are to one another using cosine similarity. The transcriptomics model benchmarking system 100 may ignore self-comparisons to not distort the results. The transcriptomics model benchmarking system 100 may then focus on the most significant relationships-those with similarity scores in the top 5% and/or bottom 5%. These may be considered predicted links of the transcriptomics machine learning model. In some embodiments, the transcriptomics model benchmarking system 100 measures model performance using a recall metric, which can determine how many of these predicted links are actual known relationships between genes. For instance, the transcriptomics model benchmarking system 100 can calculate the recall metric by comparing the predicted links with known gene-gene relationships from one or more benchmark databases (e.g., one or more curated gene/protein interaction databases).

For example, in one or more embodiments, the transcriptomics model benchmarking system 100 computes the known relationships retrieval metric by calculating pairwise cosine similarities between the aggregated perturbation embeddings of all perturbed genes. In some cases, self-links (e.g., similarities of a gene with itself) may be excluded because the self-links may distort the results (e.g., since their similarity is one). In the same or other embodiments, the transcriptomics model benchmarking system 100 next selects relationships with cosine similarities falling below the 5th percentile and/or above the 95th percentile as “predicted links.”

The transcriptomics model benchmarking system 100 may then compute a recall metric by comparing these predicted links with known gene-gene relationships from one or more benchmark databases (e.g., one or more curated gene/protein interaction databases). For each database, the recall metric can be defined as the proportion of true relationships (e.g., known links) retrieved by the model, relative to all possible gene-gene pairs in that database that are also present in the perturbation dataset. This adjustment can ensure fairness when comparing datasets with different numbers of genes. In some cases, the recall values are then multiplied by one hundred to express them as percentages (e.g., to help make the results easier to interpret).

As further illustrated in FIG. 3B, and as mentioned above, the transcriptomics model benchmarking system 100 can generate the linear interpretability of latent space metric 312. To elaborate, it can be important to interpret representations of a transcriptomics machine learning model in terms of actual gene activity (e.g., to better understand whether its internal latent space truly reflects real gene activity). For instance, to better understand the biological basis for predictions and discoveries of the transcriptomics machine learning model, the transcriptomics model benchmarking system 100 can reconstruct (and/or decode) the latent space back into one or more transcriptomic profiles (e.g., gene expression profiles). To fairly evaluate how accurately the latent embeddings can be reconstructed (and/or decoded) into the one or more transcriptomic profiles (e.g., predicted transcriptomic profiles), the transcriptomics model benchmarking system 100 can utilize a neural network (e.g., an MLP) on top of a frozen model to map the latent space back to gene expression counts.

In one or more embodiments, the transcriptomics model benchmarking system 100 assesses the quality of the reconstruction using a Spearman correlation metric between true expressions (e.g., observed transcriptomic profiles) and reconstructed expressions (e.g., predicted transcriptomic profiles). In particular, the Spearman correlation metric can measure the strength and direction of a relationship between sets of values (e.g., between the true expressions and the reconstructed expressions) based on their rankings rather than their exact values. In some cases, a high Spearman correlation indicates that the reconstructed expressions closely follow the rankings of the true expressions, even if the exact values are not identical. The transcriptomics model benchmarking system 100 can utilize alternative metrics, such as mean squared error (MSE), mean absolute error (MAE), and/or Pearson correlation. These metrics can provide insight into how well the latent space reflects true gene expression patterns.

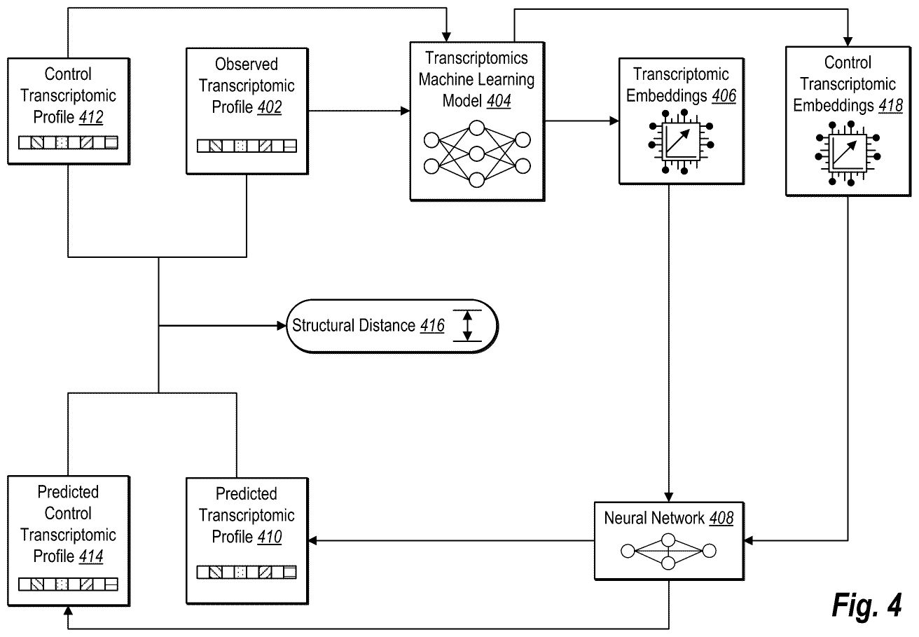

Additionally, as illustrated in FIG. 3B and as mentioned above, the transcriptomics model benchmarking system 100 can generate the structural integrity metric 314. In particular, the transcriptomics model benchmarking system 100 can generate a novel structural integrity metric to assess how well embeddings generated by a transcriptomics machine learning model preserve the relationship between control and perturbation conditions within each biological batch in the gene activity dimension. Assessing this can be crucial for utilizing predicted transcriptomic profiles (e.g., reconstructed gene expression profiles) to, for example, study gene expression changes under different conditions. See above and below (e.g., FIGS. 4-6) for additional detail regarding how the transcriptomics model benchmarking system 100 can generate the structural integrity metric.

In one or more embodiments, the transcriptomics model benchmarking system 100 can generate an evaluation framework of the one or more transcriptomic evaluation metrics 300. Specifically, the transcriptomics model benchmarking system 100 can generate the evaluation framework to assess a machine learning model (e.g., the transcriptomics machine learning model 104 and, optionally, one or more other machine learning models) in a clear and systematic way. In at least one embodiment, the transcriptomics model benchmarking system 100 generates, as part of the evaluation framework, a structured hierarchy of the one or more transcriptomic evaluation metrics 300. In some cases, when assessing the machine learning model, the transcriptomics model benchmarking system 100 can utilize the structured hierarchy to progress through the one or more transcriptomic evaluation metrics 300 in a predetermined order and/or according to a particular weighted hierarchy.

The predetermined order can, for example, ensure that fundamental criteria are met first before moving on to more specific or complex tasks. As an example, the transcriptomics model benchmarking system 100 utilizes the structured hierarchy to progress through the one or more transcriptomic evaluation metrics 300 in the following order: first, the batch effect metric 302; second, the latent space linear separability metric 304; third, the perturbation consistency metric 306; fourth, the transcriptomic evaluation metric 308; fifth, the zero-shot retrieval metric 310; sixth, the linear interpretability of latent space metric 312; and seventh, the structural integrity metric 314. In some embodiments, the transcriptomics model benchmarking system 100 utilizes the structured hierarchy to progress through the one or more transcriptomic evaluation metrics 300 in different order than what is shown above. Indeed, the transcriptomics model benchmarking system 100 can progress through the one or more transcriptomic evaluation metrics 300 following an alternative order (e.g., sapping the 2^{nd} and 3^{rd}, 4^{th} or 5^{th}, or other metrics from the hierarchical list articulated above).

In some implementations, the transcriptomics model benchmarking system 100 utilizes the structured hierarchy to apply weights in a hierarchical order (or hierarchal emphasis). To elaborate, the transcriptomics model benchmarking system 100 can apply a greater or lesser weight to each of the one or more transcriptomic evaluation metrics 300 based on a predetermined order. As an example, the predetermined order can be based on the above-mentioned order or an alternative order. For example, the transcriptomics model benchmarking system 100 can generate a benchmark that reflects a combination of a two or more of the foregoing metrics. The transcriptomics model benchmarking system 100 can combine these metrics using a weighted combination approach. For example, the transcriptomics model benchmarking system 100 can normalize each of them metrics (e.g., to a value between 0 and 1), and then combine the metrics with weights corresponding to their hierarchical order (e.g., the first metric in the hierarchy getting the highest weight, the second metric in the hierarchy getting the next highest weight, and so on for the remainder of the metrics).

As expressed above, in some embodiments, the transcriptomics model benchmarking system 100 can generate a structural integrity metric. In particular, the transcriptomics model benchmarking system 100 can generate the structural integrity metric by generating a structural distance and comparing it to a threshold (e.g., maximum) structural distance. FIG. 4 illustrates an example diagram of generating the structural integrity metric by generating the structural distance for a transcriptomics machine learning model in accordance with one or more embodiments.

As illustrated in FIG. 4, the transcriptomics model benchmarking system 100 generates one or more observed transcriptomic profiles 402. In particular, the transcriptomics model benchmarking system 100 can generate the one or more observed transcriptomic profiles 402 by creating a quantitative representation of gene expression for a perturbed cell. For instance, the transcriptomics model benchmarking system 100 can generate the one or more observed transcriptomic profiles 402 in a manner similar to what is described above in relation to FIG. 2.

As also shown in FIG. 4, the transcriptomics model benchmarking system 100 utilizes a transcriptomics machine learning model 404 (e.g., a transcriptomics foundations model) to generate transcriptomic embeddings 406 from the one or more observed transcriptomic profiles 402. For instance, the transcriptomics model benchmarking system 100 generates the transcriptomic embeddings 406 utilizing the transcriptomics machine learning model 404 in manner similar to what is described above in relation to FIG. 2.

As further shown in FIG. 4, the transcriptomics model benchmarking system 100 can utilize a neural network 408 to generate one or more predicted transcriptomic profiles 410. In particular, the transcriptomics model benchmarking system 100 can utilize the neural network 408 (e.g., a multilayer perceptron (MLP)) to reconstruct the one or more predicted transcriptomic profiles 410 (e.g., gene expression profiles) for the cells exposed to perturbations from the transcriptomic embeddings 406. For instance, the transcriptomics model benchmarking system 100 can utilize the neural network 408 (e.g., the MLP) on top of a frozen model to reconstruct, decode, and/or map the transcriptomic embeddings 406 back to gene expression counts for cells exposed to perturbations. This process can, for example, enable the transcriptomics model benchmarking system 100 to predict gene expression changes based on the perturbation effects captured in the transcriptomic embeddings 406.

As shown in FIG. 4, the transcriptomics model benchmarking system 100 generates a structural distance 416 for the transcriptomics machine learning model 404. In particular, the transcriptomics model benchmarking system 100 can generate the structural distance 416 for the transcriptomics machine learning model 404 from the one or more predicted transcriptomic profiles 410 and the one or more observed transcriptomic profiles 402 relative to control transcriptomic profiles across batches. For instance, the transcriptomics model benchmarking system 100 applies at least one control transcriptomic profile 412 to the one or more observed transcriptomic profiles 402 to generate one or more adjusted observed transcriptomic profiles. In the same or other embodiments, the transcriptomics model benchmarking system 100 applies at least one predicted control transcriptomic profile 414 to the one or more predicted transcriptomic profiles 410 to generate one or more adjusted predicted transcriptomic profiles. Upon applying the at least one control transcriptomic profile 412 and the at least one predicted control transcriptomic profile 414, the transcriptomics model benchmarking system 100 can generate the structural distance 416 by comparing the one or more adjusted observed transcriptomic profiles to the one or more adjusted predicted transcriptomic profiles.

In one or more embodiments, the transcriptomics model benchmarking system 100 generates the at least one predicted control transcriptomic profile 414 from the at least one control transcriptomic profile 412. Specifically, the transcriptomics model benchmarking system 100 can utilize the transcriptomics machine learning model 404 to generate control transcriptomic embeddings 418 from the at least one control transcriptomic profile 412 (e.g., in a manner similar to what is described above in relation to the one or more observed transcriptomic profiles 402). In some embodiments, the transcriptomics model benchmarking system 100 can utilize the neural network 408 to reconstruct the at least one predicted control transcriptomic profile 414 from the control transcriptomic embeddings 418 (e.g., in a manner similar to what is described above in relation to the one or more predicted transcriptomic profiles 410).

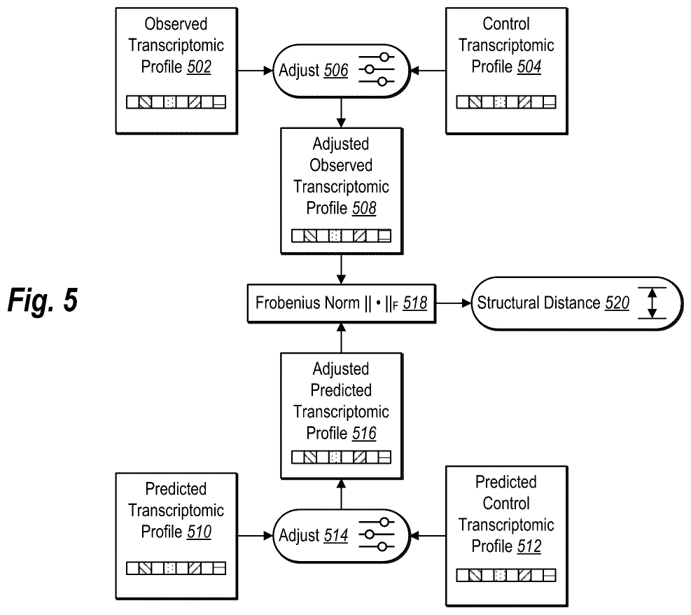

As expressed above, in some embodiments, the transcriptomics model benchmarking system 100 can generate a structural integrity metric. In particular, the transcriptomics model benchmarking system 100 can generate the structural integrity metric by generating a structural distance and comparing it to a threshold (e.g., maximum) structural distance. FIG. 5 illustrates an example diagram of generating a structural distance by comparing adjusted observed transcriptomic profiles and adjusted predicted transcriptomic profiles in accordance with one or more embodiments.

As illustrated in FIG. 5, the transcriptomics model benchmarking system 100 performs an act 506 to adjust (or modify) an observed transcriptomic profile 502. In particular, the transcriptomics model benchmarking system 100 can perform the act 506 by applying a control transcriptomic profile 504 to the observed transcriptomic profile 502 to, for example, account for batch specific variability. For instance, the transcriptomics model benchmarking system 100 performs the act 506 by subtracting a corresponding control profile (e.g., the control transcriptomic profile 504) from the observed transcriptomic profile 502. As an example, the transcriptomics model benchmarking system 100 subtracts an expression level in the control transcriptomic profile 504 from an expression level in the observed transcriptomic profile 502 for each gene in a sample.

As also illustrated in FIG. 5, the transcriptomics model benchmarking system 100 generates an adjusted observed transcriptomic profile 508. In particular, upon performing the act 506, the transcriptomics model benchmarking system 100 generates the adjusted observed transcriptomic profile 508. In one or more embodiments, by performing the act 506, the transcriptomics model benchmarking system 100 centers (or aligns) the observed transcriptomic profile 502 to a baseline (e.g., the control transcriptomic profile 504). In some cases, this centering accounts for batch-specific variability and focuses on the perturbation effects.

As further illustrated in FIG. 5, the transcriptomics model benchmarking system 100 performs an act 514 to adjust (or modify) a predicted transcriptomic profile 510. In particular, the transcriptomics model benchmarking system 100 can perform the act 514 by applying a predicted control transcriptomic profile 512 to the predicted transcriptomic profile 510 to, for example, account for batch specific variability. For instance, the transcriptomics model benchmarking system 100 performs the act 514 by subtracting a corresponding control profile (e.g., the predicted control transcriptomic profile 512, which can be the same as the control transcriptomic profile 504) from the predicted transcriptomic profile 510. As an example, the transcriptomics model benchmarking system 100 subtracts an expression level in the predicted control transcriptomic profile 512 from an expression level in the predicted transcriptomic profile 510 for each gene in a sample.

As shown in FIG. 5, the transcriptomics model benchmarking system 100 generates an adjusted predicted transcriptomic profile 516. In particular, upon performing the act 514, the transcriptomics model benchmarking system 100 generates the adjusted predicted transcriptomic profile 516. In one or more embodiments, by performing the act 514, the transcriptomics model benchmarking system 100 centers (or aligns) the predicted transcriptomic profile 510 to a baseline (e.g., the predicted control transcriptomic profile 512). In some cases, this centering accounts for batch-specific variability and focuses on the perturbation effects.

As also shown in FIG. 5, the transcriptomics model benchmarking system 100 performs the act 518 to generate a structural distance 520. In particular, the transcriptomics model benchmarking system 100 can perform the act 518 by computing, for each batch b, the Frobenius norm of the matrix obtained by subtracting an adjusted observed transcriptomic profile gene expression matrix from an adjusted predicted transcriptomic profile gene expression matrix:

$$Structural Distance = \frac{1}{B} {\sum }_{b = 1}^{B} {‖ {\tilde{Y}}_{pred}^{( b )} - {\tilde{Y}}_{actual}^{( b )} ‖}_{F} ,$$

where B is the total number of batches,

$${\tilde{Y}}_{pred}^{( b )} and {\tilde{Y}}_{actual}^{( b )}$$

are the adjusted predicted adjusted predicted transcriptomic profile and adjusted observed transcriptomic profile gene expression matrices for batch b, respectively, and ∥·∥_{F} denotes the Frobenius norm.

In the same or other embodiments, the transcriptomics model benchmarking system 100 generates a structural distance for one or more batches. As an example, the transcriptomics model benchmarking system 100 can generate a first structural distance based on a first batch with a first set of controls and a second structural distance based on a second batch with a second set of controls. Indeed, the transcriptomics model benchmarking system 100 can generate structural distance metrics based for a variety of different batches with a variety of different controls. In some cases, the transcriptomics model benchmarking system 100 can generate a combined structural distance by combining the first structural distance and the second structural distance. Further, the transcriptomics model benchmarking system 100 can, in some embodiments, generate the combined structural distance by combining the first structural distance and the second structural distance with other generated structural distance metrics for different batches.

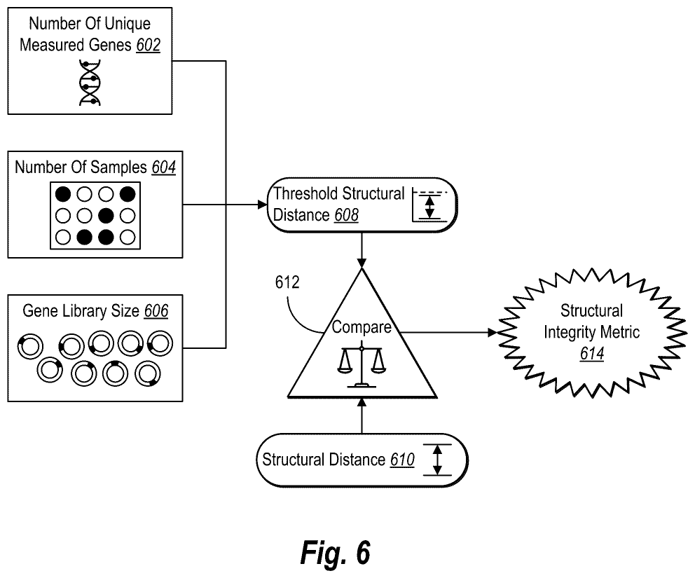

As expressed above, in some embodiments, the transcriptomics model benchmarking system 100 can generate a structural integrity metric. In particular, the transcriptomics model benchmarking system 100 can generate the structural integrity metric by generating a structural distance and comparing it to a threshold (e.g., maximum) structural distance. FIG. 6 illustrates an example diagram of generating the structural integrity metric by comparing the structural distance to the threshold structural distance in accordance with one or more embodiments.

As illustrated in FIG. 6, the transcriptomics model benchmarking system 100 determines a threshold structural distance 608 (e.g., a maximum threshold structural distance). For instance, the threshold structural distance 608 can be the theoretical upper bound for a generated structural distance based on a number of unique measured genes 602 in one or more observed transcriptomic profiles, a number of samples 604 in a batch, and on a gene library size 606. In particular, given the number of unique measured genes g and assuming the gene library size is M, and where no is the number of samples in a batch b, the threshold structural distance 608 can be calculated by the following equation:

$$Structural {Distance}_{max} = \frac{1}{B} {\sum }_{b = 1}^{B} M \sqrt{{n}_{b} \times g} \approx \frac{1}{B} {\sum }_{b = 1}^{B} {‖ {\tilde{Y}}_{actual}^{( b )} ‖}_{F}$$

In the same or other embodiments, the transcriptomics model benchmarking system 100 generates a threshold structural distance for one or more batches. For example, the transcriptomics model benchmarking system 100 can generate a first threshold structural distance based on first batch with a first set of controls and a second threshold structural distance based on a second batch with a second batch with a second set of controls (and a second number of samples in the second batch). Indeed, the transcriptomics model benchmarking system 100 can generate a variety of threshold structural distance metrics based on a number of batches with different sets of controls. In some cases, the transcriptomics model benchmarking system 100 can generate a combined threshold structural distance by combining the first threshold structural distance and the second threshold structural distance (e.g., by averaging, adding, or otherwise combining). Further, the transcriptomics model benchmarking system 100 can, in some embodiments, generate the combined threshold structural distance by combining the first threshold structural distance and the second threshold structural distance with other generated threshold structural distance metrics (e.g., for other batches).

As also illustrated in FIG. 6, the transcriptomics model benchmarking system 100 generates the structural integrity metric 614 by performing an act 612. In particular, the transcriptomics model benchmarking system 100 can perform the act 612 by comparing the threshold structural distance 608 and a structural distance 610 (as described above in greater detail). For instance, the transcriptomics model benchmarking system 100 performs that act 612 by taking a ratio of the structural distance 610 relative to the threshold structural distance 608. As an example, the transcriptomics model benchmarking system 100 computes the structural integrity metric 614 as:

$$Structural Integrity = 1 - \frac{Structural Distance}{Structural {Distance}_{max}}$$

In the same or other embodiments, the transcriptomics model benchmarking system 100 generates the structural integrity metric 614 by comparing combined threshold structural distance metrics for a plurality of batches and a combined structural distance for the plurality of batches (as described above in relation to FIG. 5). For instance, the transcriptomics model benchmarking system 100 performs that act 612 by taking a ratio of the combined structural distance relative to the combined threshold structural distance.

In one or more embodiments, higher values of structural integrity indicate better preservation of the structural relationships in gene expression data. Therefore, this metric can provide an assessment of how well a transcriptomics machine learning model captures the overall structure of gene expression changes while accounting for batch-specific variability.

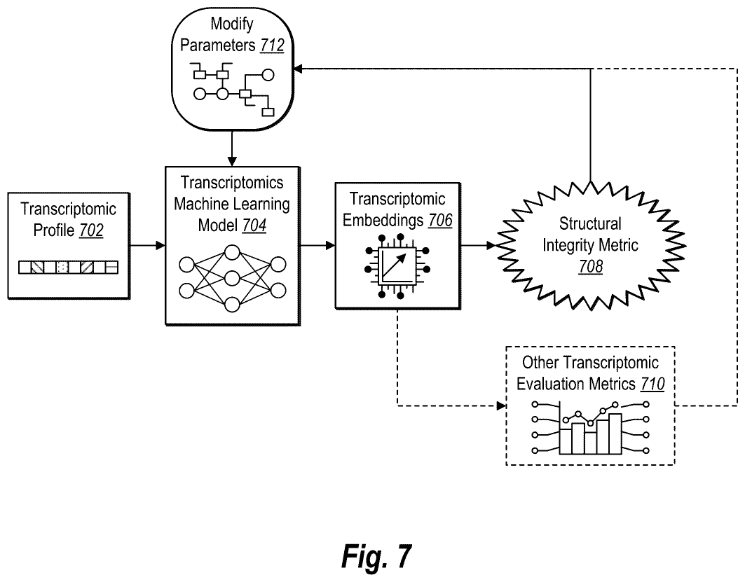

As expressed above, the transcriptomics model benchmarking system 100 can train a transcriptomics machine learning model using machine learning data associated with an evaluation framework (e.g., as a structured hierarchy) of a plurality of transcriptomic evaluation metrics. In particular, the transcriptomics model benchmarking system 100 can modify the parameters of the transcriptomics machine learning model based on machine learning data associated with the evaluation framework. FIG. 7 illustrates an example diagram of training a transcriptomics machine learning model based on machine learning data associated with a structural integrity metric and, optionally, one or more other transcriptomic metrics in accordance with one or more embodiments.

As illustrated in FIG. 7, the transcriptomics model benchmarking system 100 utilizes transcriptomics machine learning model 704 to generate transcriptomic embeddings 706. In particular, the transcriptomics model benchmarking system 100 can utilize the transcriptomics machine learning model 704 to generate the transcriptomic embeddings 706 from observed transcriptomic profiles of cells 702 exposed to perturbations, as outlined above in greater detail.

As also illustrated in FIG. 7, the transcriptomics model benchmarking system 100 generates a plurality of transcriptomic evaluation metrics which includes a structural integrity metric 708. Specifically, the transcriptomics model benchmarking system 100 can utilize the transcriptomic embeddings 706 to generate the structural integrity metric 708 and/or one or more other transcriptomic evaluation metrics 710, as outlined above (e.g., in FIG. 3).

As further illustrated in FIG. 7, the transcriptomics model benchmarking system 100 can perform an act 712 to modify parameters of (e.g., train) the transcriptomics machine learning model 704. In particular, the transcriptomics model benchmarking system 100 can perform the act 712 based on machine learning data associated the structural integrity metric 708 and, optionally, the one or more other transcriptomic evaluation metrics 710. For example, the transcriptomics model benchmarking system 100 can utilize back propagation and/or gradient descent to modify internal parameters (e.g., learned weights within layers of a neural network) to improve a measure of loss (e.g., improve the structural integrity metric 708). Indeed, by iteratively generating embeddings and applying the structural integrity metric as a measure of loss, the system can train the transcriptomics machine learning model 704 to generate improved transcriptomic embeddings.

In one or more embodiments, the transcriptomics model benchmarking system 100 performs the act 712 based on machine learning data associated with an evaluation framework (e.g., as a structured hierarchy) of the plurality of transcriptomic evaluation metrics. In some cases, by training the transcriptomics machine learning model 704 in at least one of these ways, the transcriptomics model benchmarking system 100 enables the transcriptomics machine learning model 704 to more accurately and efficiently capture biologically relevant signals.

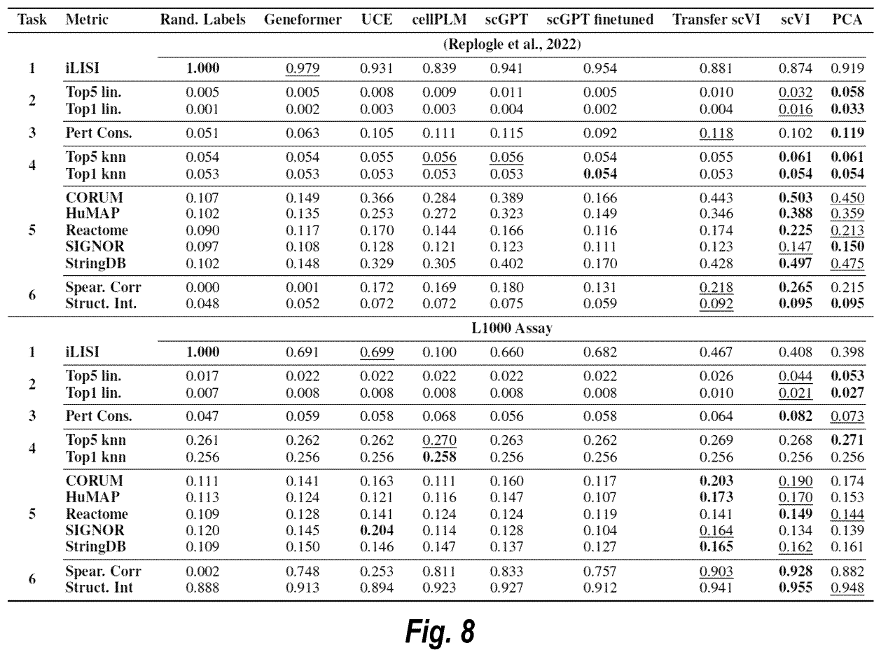

As expressed above, in some embodiments, the transcriptomics model benchmarking system 100 generates a transcriptomic benchmark to evaluate, compare, and/or validate a performance of one or more machine learning models and/or transcriptomic evaluation methods. In particular, the transcriptomics model benchmarking system 100 can combine a plurality of generated transcriptomic evaluation metrics to generate the transcriptomic benchmark. As an example, the transcriptomic benchmark 110 can include a combined benchmark score and/or a table, graph, chart, and/or other dataset. FIG. 8 illustrates an example table that summarizes a performance comparison of various transcriptomics machine learning models on various evaluation tasks and metrics using transcriptomic benchmark datasets in accordance with one or more embodiments.

As illustrated in FIG. 8, the transcriptomics model benchmarking system 100 generates a plurality of transcriptomic evaluation metrics. For instance, the transcriptomics model benchmarking system 100 can generate the plurality of transcriptomic evaluation metrics to assess: (1) data integrating and batch effect reduction (e.g., represented in the table as “iLISI”); (2) latent space linear separability of known perturbations (e.g., represented in the table as “Top5 lin.” and “Top 1 lin.”); (3) perturbation consistency (e.g., represented in the table as “Pert Cons.”); (4) latent space direct organization (e.g., represented in the table as “Top5 knn” and “Top1 knn”); (5) zero-shot retrieval of known biological relationships (e.g., represented in the table as “CORUM,” “HuMAP,” “Reactome,” “SIGNOR,” and “StringDB); (6) linear interpretability of the latent space (e.g., represented in the table as “Spear. Corr”); and/or (7) structural integrity (e.g., represented in the table as “Struct. Int.”).

As also illustrated in FIG. 8, the transcriptomics model benchmarking system 100 combines the plurality of transcriptomic evaluation metrics to generate one or more transcriptomic benchmarks. For instance, as shown in FIG. 8, the transcriptomics model benchmarking system 100 can combine the plurality of transcriptomic evaluation metrics to generate one or more transcriptomic benchmark datasets. As shown, for example, the transcriptomics model benchmarking system 100 can generate a transcriptomic benchmark dataset for Replogle (e.g., a single-cell gene knockout dataset) and L1000 CRISPR Assay (e.g., a bulk RNA dataset). Further, each transcriptomic evaluation metrics (e.g., scores) can be the average of one or more runs. As an example, the transcriptomic evaluation metrics (e.g., scores) shown in FIG. 8 are the average of five different runs.

As further represented in FIG. 8, the transcriptomics model benchmarking system 100 can generate the one or more transcriptomic benchmarks to evaluate, compare, and/or validate the performance of one or more transcriptomics machine learning models and/or one or more transcriptomic evaluation methods in analyzing transcriptomic data (e.g., perturbations). For instance, as shown in FIG. 8, the transcriptomics model benchmarking system 100 evaluates, compares, and/or validates the performance transcriptomics machine learning models (e.g., transcriptomics foundation models), including scVI, Geneformer, UCE, cellPLM, scGPT, and scGPT finetuned. As further shown in FIG. 8, the transcriptomics model benchmarking system 100 evaluates, compares, and/or validates the performance of transcriptomic evaluation methods, including methods or techniques of learning from transcriptomics data such as scVI (or a variational autoencoder that can be tailored for single-cell RNA sequencing), Transfer scVI, and PCA. The transcriptomics model benchmarking system 100 may also utilize random labels (e.g., “Rand. Labels”) to serve as a baseline comparison (e.g., to represent the performance of a model or task when labels are assigned randomly).

As shown in FIG. 8, current foundation models may not generalize well to perturbation-related tasks compared to simpler approaches like PCA and scVI. In particular, PCA, which can be applied on raw gene counts, and scVI, which can be trained from scratch on the same dataset, can be shown to consistently outperform foundation models across most tasks, except for batch effect reduction (e.g., Task 1). Notably, scVI can be shown to achieve strong performance in both scenarios: when trained directly on the evaluation dataset (scVI) and when used in a zero-shot transfer learning context (Transfer scVI), in which it can be pre-trained on a different cell line, perturbation type, and sequencing technique before being evaluated on Replogle and L1000 data. In some cases, Transfer sc VI ranks third overall, highlighting its robustness in handling strong out of distribution. Further, in some cases, Transfer scVI consistently surpasses sc VI for batch effect reduction, as this metric is easily optimized by capturing higher levels of noise, which is the case for transfer learning zero shot applications. Additionally, in some instances, PCA shows better structural integrity than Transfer scVI for L1000 assay, while Transfer scVI is better at reconstructing expression counts. In some embodiments, Transfer scVI can preserve bias from training data structure, while PCA can conserve current data structure integrity.

As also shown in FIG. 8, foundation models such as Geneformer and scGPT show competitive performance only in batch effect reduction, where random embeddings achieve near-optimal results, but they struggle across more biologically meaningful tasks. This can suggest that their training objectives, which likely focus on reducing batch effects, are insufficient for capturing nuanced biological insights required for perturbation tasks. In some instances, finetuning scGPT on the same evaluation data improves its performance on batch effect reduction but has little to no effect on linear separability of perturbations (e.g., Task 2) and dramatically reduces performance on zero-shot recall of known biological relationships (e.g., Task 5). This may hint that its learning objective may not be adapted to learning relevant representations of perturbation biology even when trained on its evaluation data. In one or more embodiments, the overall results shown in FIG. 8 indicate that while foundation models can be tuned for specific technical metrics, they do not yet effectively generalize to biologically complex tasks like perturbation analysis, where scVI and PCA may remain more reliable.

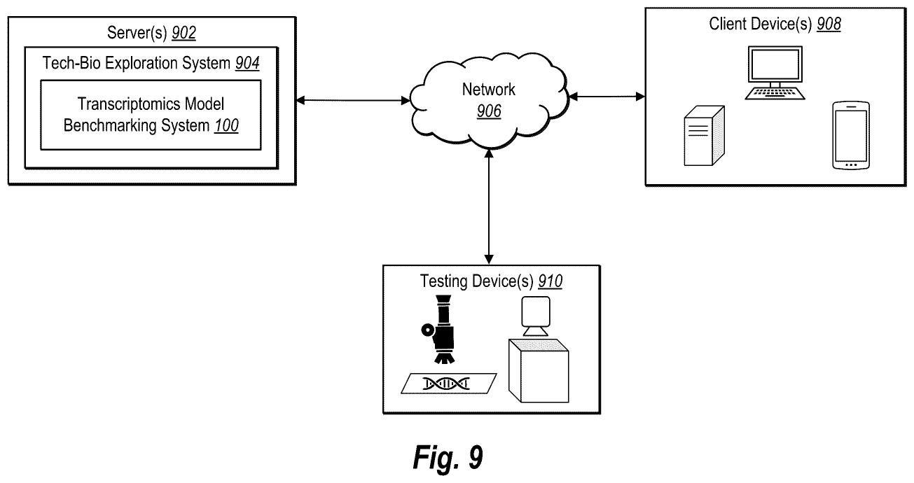

In some embodiments, the transcriptomics model benchmarking system 100 is part of a networking environment. For example, FIG. 9 illustrates a diagram of an example environment in which the transcriptomics model benchmarking system 100 can operate in accordance with one or more embodiments.

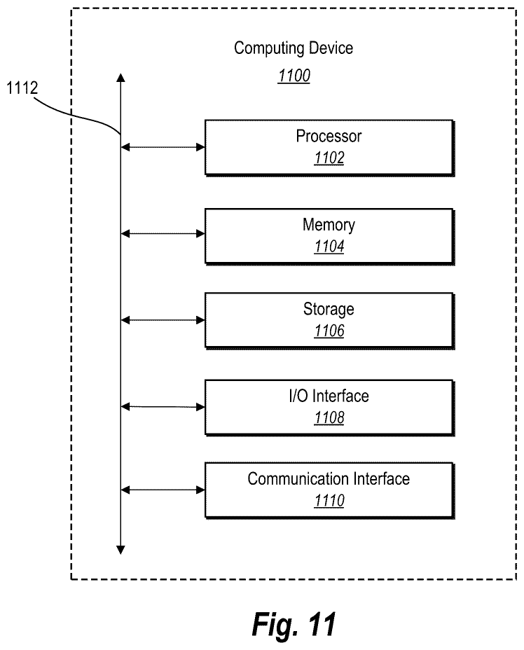

As shown in FIG. 9, the environment includes server(s) 902 (which includes a tech-bio exploration system 904 and the transcriptomics model benchmarking system 100), a network 906, client device(s) 908, and testing device(s) 910. As further illustrated in FIG. 9, the various computing devices within the environment can communicate via the network 906. Although FIG. 9 illustrates the transcriptomics model benchmarking system 100 being implemented by a particular component and/or device within the environment, the transcriptomics model benchmarking system 100 can be implemented, in whole or in part, by other computing devices and/or components in the environment (e.g., the client device(s) 908). Additional description regarding the illustrated computing devices is provided with respect to FIG. 11 below.

As shown in FIG. 9, the server(s) 902 can include the tech-bio exploration system 904. In some embodiments, the tech-bio exploration system 904 can determine, store, generate, analyze and/or display tech-bio information including maps of biology, biology experiments from various sources, and/or machine learning tech-bio predictions. For instance, the tech-bio exploration system 904 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). In one or more embodiments, the server(s) 902 comprises a data server. In some implementations, the server(s) 902 comprises a communication server or a web-hosting server.

Further, the tech-bio exploration system 904 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 904 can generate or determine a variety of predictions and inter-relationships for improving treatments/interventions.

To illustrate, the tech-bio exploration system 904 can generate maps of biology indicating biological inter-relationships or similarities between these various input signals to discover potential new treatments. For example, the tech-bio exploration system 904 can utilize machine learning and/or maps of biology to identify a similarity between a first gene associated with disease treatment and a second gene previously unassociated with the disease based on a similarity in resulting phenotypes from gene knockout experiments. The tech-bio exploration system 904 can then identify new treatments based on the gene similarity (e.g., by targeting compounds the impact the second gene). Similarly, the tech-bio exploration system 904 can analyze signals from a variety of sources (e.g., protein interactions, or in vivo experiments) to predict efficacious treatments based on various levels of biological data.

The tech-bio exploration system 904 can generate GUIs comprising dynamic user interface elements to convey tech-bio information and receive user input for intelligently exploring tech-bio information. Indeed, as mentioned above, the tech-bio exploration system 904 can generate GUIs displaying different maps of biology that intuitively and efficiently express complex interactions between different biological systems for identifying improved treatment solutions. Furthermore, the tech-bio exploration system 904 can also electronically communicate tech-bio information between various computing devices.

As shown in FIG. 9, the tech-bio exploration system 904 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 904 collects, manages, and transmits data across a variety of different entities, accounts, and devices. In some cases, the tech-bio exploration system 904 is a network system that facilitates access to (and analysis of) tech-bio information within a centralized operating system. Indeed, the tech-bio exploration system 904 can link data from different network-based research institutions to generate and analyze maps of biology.

As shown in FIG. 9, the tech-bio exploration system 904 can include a system that comprises the transcriptomics model benchmarking system 100 that generates, stores, manages, transmits, and analyzes cell and subject perturbation datasets. For example, the transcriptomics model benchmarking system 100 can generate perturbation experiment unit embeddings utilizing a machine learning model and synthesize the embeddings according to various filtration, alignment, and aggregation models. Further, the transcriptomics model benchmarking system 100 can identify similarity measures between aggregated perturbation embeddings (e.g., perturbation-level embeddings) of a perturbation embedding model and determine a benchmark measure for the perturbation embedding model. For example, the transcriptomics model benchmarking system 100 can generate a transcriptomic benchmark for the perturbation experiment unit embeddings of the perturbation embedding model and/or a transcriptomic benchmark for the identified similarity measures for display.

As also illustrated in FIG. 9, the environment includes the client device(s) 908. For example, the client device(s) 908 may include, but is not limited to, a mobile device (e.g., smartphone, tablet) or other type of computing device, including those explained below with reference to FIG. 11. Additionally, the client device(s) 908 can include a computing device associated with (and/or operated by) user accounts for the tech-bio exploration system 904. Moreover, the environment can include various numbers of client devices that communicate and/or interact with the tech-bio exploration system 904 and/or the transcriptomics model benchmarking system 100.

Furthermore, in one or more implementations, the client device(s) 908 includes a client application. The client application can include instructions that (upon execution) cause the client device(s) 908 to perform various actions. For example, a user of a user account can interact with the client application on the client device(s) 908 to access tech-bio information, initiate a request for a benchmark measure and/or generate GUIs comprising similarity measures, benchmark measures, or other machine learning dataset and/or machine learning predictions/results.

As further shown in FIG. 9, the environment includes the network 906. As mentioned above, the network 906 can enable communication between components of the environment. In one or more embodiments, the network 906 may include a suitable network and may communicate using a various number of communication platforms and technologies suitable for transmitting data and/or communication signals, examples of which are described with reference to FIG. 11. Furthermore, although FIG. 9 illustrates computing devices communicating via the network 906, the various components of the environment can communicate and/or interact via other methods (e.g., communicate directly).

As mentioned previously, in one or more implementations, the transcriptomics model benchmarking system 100 generates and accesses machine learning objects, such as results from biological assays, in vivo trials, results from perturbation embedding models, etc. As shown, in FIG. 9, the transcriptomics model benchmarking system 100 can communicate with testing device(s) 910 to obtain and then store this information. For example, the tech-bio exploration system 904 can interact with the testing device(s) 910 that include intelligent robotic devices and camera devices for generating and capturing digital images of cellular phenotypes resulting from different perturbations (e.g., genetic knockouts or compound treatments of stem cells). Similarly, the testing device(s) can include camera devices and/or other sensors (e.g., heat or motion sensors) capturing real-time information from animals as part of in vivo experimentation. The tech-bio exploration system 904 can also interact with a variety of other testing device(s) such as devices for determining, generating, or extracting gene sequences or protein information.

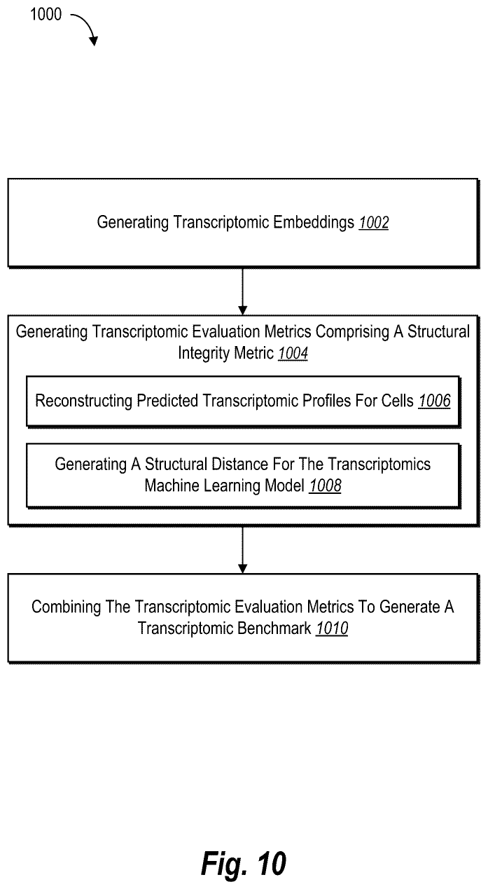

FIGS. 1-9, the corresponding text, and the examples provide a number of different systems and methods for benchmarking transcriptomics machine learning models for perturbation analysis. In addition to the foregoing, implementations can also be described in terms of flowcharts comprising acts steps in a method for accomplishing a particular result. For example, FIG. 10 illustrates an example flowchart of a series of acts for generating a transcriptomic benchmark in accordance with one or more embodiments. While FIG. 10 illustrates acts according to certain implementations, alternative implementations may omit, add to, reorder, and/or modify any of the acts shown in FIG. 10. The acts of FIG. 10 can be performed as part of a method. Alternatively, a non-transitory computer readable medium can comprise instructions that, when executed by one or more processors, cause a computing device to perform the acts of FIG. 10. In still further implementations, a system can perform the acts of FIG. 10. Additionally, the acts described herein may be repeated or performed in parallel with one another or in parallel with different instances of the same or other similar acts.

As illustrated in FIG. 10, the series of acts 1000 may include an act 1002 of generating transcriptomic embeddings. In particular, the act 1002 involves generating, utilizing a transcriptomics machine learning model, transcriptomic embeddings from observed transcriptomic profiles of cells exposed to perturbations. The series of acts 1000 can also include an act 1004 of generating transcriptomic evaluation metrics comprising a structural integrity metric. In particular, the act 1004 can involve generating, utilizing the transcriptomic embeddings, a plurality of transcriptomic evaluation metrics comprising a structural integrity metric by an act 1006 and an act 1008. For instance, the series of acts 1000 can include the act 1006 of reconstructing predicted transcriptomic profiles for cells. In particular, the act 1006 can involve reconstructing, utilizing a neural network, predicted transcriptomic profiles for the cells exposed to the perturbations from the transcriptomic embeddings. Further, the series of acts 1000 can include the act 1008 of generating a structural distance for the transcriptomics machine learning model. In particular, the act 1008 can involve generating a structural distance for the transcriptomics machine learning model from the predicted transcriptomic profiles and the observed transcriptomic profiles relative to control transcriptomic profiles across batches. Moreover, the series of acts 1000 can include an act 1010 of combining the transcriptomic evaluation metrics to generate a transcriptomic benchmark. In particular, the act 1010 can involve combining the plurality of transcriptomic evaluation metrics comprising the structural integrity metric to generate a transcriptomic benchmark for evaluating the transcriptomics machine learning model.

In some embodiments, the series of acts 1000 includes an act of generating the plurality of transcriptomic evaluation metrics by: generating a batch effect metric by comparing sets of transcriptomic embeddings across batches within an embedding feature space; and generating at least one of: a latent space linear separability metric; a perturbation consistency metric; a latent space direct organization metric; or a zero-shot retrieval metric. The series of acts 1000 can also include an act of generating an additional plurality of transcriptomic evaluation metrics comprising an additional structural integrity metric for an additional transcriptomics machine learning model; and combining the additional plurality of transcriptomic evaluation metrics comprising the additional structural integrity metric to generate an additional transcriptomic benchmark for comparing the transcriptomics machine learning model and the additional transcriptomics machine learning model.

In some embodiments, the series of acts 1000 includes an act of generating adjusted observed transcriptomic profiles by modifying the observed transcriptomic profiles with the control transcriptomic profiles of the batches; and generating adjusted predicted transcriptomic profiles by modifying the predicted transcriptomic profiles with the control transcriptomic profiles of the batches. In the same or other embodiments, the series of acts 1000 includes an act of generating the structural distance by comparing the adjusted observed transcriptomic profiles and the adjusted predicted transcriptomic profiles.

In one or more embodiments, the series of acts 1000 includes an act of determining a threshold structural distance based on a number of measured genes in the observed transcriptomic profiles and a number of samples in a batch. The series of acts 1000 can also include an act of generating the structural integrity metric by comparing the structural distance and the threshold structural distance.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 11 illustrates a block diagram of an example computing device 1100 that may be configured to perform one or more of the processes described above. One will appreciate that one or more computing devices, such as the computing device 1100 may represent the computing devices described above. In one or more embodiments, the computing device 1100 may be a mobile device (e.g., a mobile telephone, a smartphone, a PDA, a tablet, a laptop, a camera, a tracker, a watch, a wearable device, etc.). In some embodiments, the computing device 1100 may be a non-mobile device (e.g., a desktop computer or another type of client device). Further, the computing device 1100 may be a server device that includes cloud-based processing and storage capabilities.

As shown in FIG. 11, the computing device 1100 can include one or more processor(s) 1102, memory 1104, a storage device 1106, input/output interfaces 1108 (or “I/O interfaces 1108”), and a communication interface 1110, which may be communicatively coupled by way of a communication infrastructure (e.g., bus 1112). While the computing device 1100 is shown in FIG. 11, the components illustrated in FIG. 11 are not intended to be limiting. Additional or alternative components may be used in other embodiments. Furthermore, in certain embodiments, the computing device 1100 includes fewer components than those shown in FIG. 11. Components of the computing device 1100 shown in FIG. 11 will now be described in additional detail.

In particular embodiments, the processor(s) 1102 includes hardware for executing instructions, such as those making up a computer program. As an example, and not by way of limitation, to execute instructions, the processor(s) 1102 may retrieve (or fetch) the instructions from an internal register, an internal cache, memory 1104, or a storage device 1106 and decode and execute them.

The computing device 1100 includes memory 1104, which is coupled to the processor(s) 1102. The memory 1104 may be used for storing data, metadata, and programs for execution by the processor(s). The memory 1104 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1104 may be internal or distributed memory.

The computing device 1100 includes a storage device 1106 includes storage for storing data or instructions. As an example, and not by way of limitation, the storage device 1106 can include a non-transitory storage medium described above. The storage device 1106 may include a hard disk drive (HDD), flash memory, a Universal Serial Bus (USB) drive or a combination these or other storage devices.

As shown, the computing device 1100 includes one or more I/O interfaces 1108, which are provided to allow a user to provide input to (such as user strokes), receive output from, and otherwise transfer data to and from the computing device 1100. These I/O interfaces 1108 may include a mouse, keypad or a keyboard, a touch screen, camera, optical scanner, network interface, modem, other known I/O devices or a combination of such I/O interfaces 1108. The touch screen may be activated with a stylus or a finger.

The I/O interfaces 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1108 are configured to provide graphical data to a display for presentation to a user. The graphical data may be representative of one or more graphical user interfaces and/or any other graphical content as may serve a particular implementation.

The computing device 1100 can further include a communication interface 1110. The communication interface 1110 can include hardware, software, or both. The communication interface 1110 provides one or more interfaces for communication (such as, for example, packet-based communication) between the computing device and one or more other computing devices or one or more networks. As an example, and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI. The computing device 1100 can further include a bus 1112. The bus 1112 can include hardware, software, or both that connects components of computing device 1100 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1100 can include a client device that includes a requester application or a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 904 may include a variety of servers, sub-systems, programs, modules, logs, and data stores. In particular embodiments, the tech-bio exploration system 904 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 904 may also include suitable components such as network interfaces, security mechanisms, load balancers, failover servers, management-and-network-operations consoles, other suitable components, or any suitable combination thereof. In particular embodiments, the tech-bio exploration system 104 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 904 and one or more client devices. An action logger may be used to receive communications from a web server about a user's actions on or off the tech-bio exploration system 904. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 104. A privacy setting of a user determines how particular information associated with a user can be shared. The authorization server may allow users to opt in to or opt out of having their actions logged by the tech-bio exploration system 104 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a transcriptomics machine learning model, transcriptomic embeddings from observed transcriptomic profiles of cells exposed to perturbations;
generating, utilizing the transcriptomic embeddings, a plurality of transcriptomic evaluation metrics comprising a structural integrity metric by:
reconstructing, utilizing a neural network, predicted transcriptomic profiles for the cells exposed to the perturbations from the transcriptomic embeddings; and
generating a structural distance for the transcriptomics machine learning model from the predicted transcriptomic profiles and the observed transcriptomic profiles relative to control transcriptomic profiles across batches; and
combining the plurality of transcriptomic evaluation metrics comprising the structural integrity metric to generate a transcriptomic benchmark for evaluating the transcriptomics machine learning model.

2. The computer-implemented method of claim 1, wherein generating the plurality of transcriptomic evaluation metrics comprises:
generating a batch effect metric by comparing sets of transcriptomic embeddings across batches within an embedding feature space; and
generating at least one of: a latent space linear separability metric; a perturbation consistency metric; a latent space direct organization metric; or a zero-shot retrieval metric.

3. The computer-implemented method of claim 1, further comprising:
generating an additional plurality of transcriptomic evaluation metrics comprising an additional structural integrity metric for an additional transcriptomics machine learning model; and
combining the additional plurality of transcriptomic evaluation metrics comprising the additional structural integrity metric to generate an additional transcriptomic benchmark for comparing the transcriptomics machine learning model and the additional transcriptomics machine learning model.

4. The computer-implemented method of claim 1, further comprising:
generating adjusted observed transcriptomic profiles by modifying the observed transcriptomic profiles with the control transcriptomic profiles of the batches; and
generating adjusted predicted transcriptomic profiles by modifying the predicted transcriptomic profiles with predicted control transcriptomic profiles of the batches generated from the control transcriptomic profiles.

5. The computer-implemented method of claim 4, further comprising generating the structural distance by comparing the adjusted observed transcriptomic profiles and the adjusted predicted transcriptomic profiles.

6. The computer-implemented method of claim 1, further comprising determining a threshold structural distance based on a number of measured genes in the observed transcriptomic profiles and a number of samples in a batch.

7. The computer-implemented method of claim 6, further comprising generating the structural integrity metric by comparing the structural distance and the threshold structural distance.

8. A system comprising:
at least one processor; and
at least one non-transitory computer-readable storage medium storing instructions that, when executed by the at least one processor, cause the system to:
generate, utilizing a transcriptomics machine learning model, transcriptomic embeddings from observed transcriptomic profiles of cells exposed to perturbations;
generate, utilizing the transcriptomic embeddings, a plurality of transcriptomic evaluation metrics comprising a structural integrity metric by:
reconstructing, utilizing a neural network, predicted transcriptomic profiles for the cells exposed to the perturbations from the transcriptomic embeddings; and
generating a structural distance for the transcriptomics machine learning model from the predicted transcriptomic profiles and the observed transcriptomic profiles relative to control transcriptomic profiles across batches; and
combine the plurality of transcriptomic evaluation metrics comprising the structural integrity metric to generate a transcriptomic benchmark for evaluating the transcriptomics machine learning model.

9. The system of claim 8, further comprising instructions that, when executed by the at least one processor, cause the system to generate the plurality of transcriptomic evaluation metrics by:
generating a batch effect metric by comparing sets of transcriptomic embeddings across batches within an embedding feature space; and
generating at least one of: a latent space linear separability metric; a perturbation consistency metric; a latent space direct organization metric; or a zero-shot retrieval metric.

10. The system of claim 8, further comprising instructions that, when executed by the at least one processor, cause the system to:
generate an additional plurality of transcriptomic evaluation metrics comprising an additional structural integrity metric for an additional transcriptomics machine learning model; and
combine the additional plurality of transcriptomic evaluation metrics comprising the additional structural integrity metric to generate an additional transcriptomic benchmark for comparing the transcriptomics machine learning model and the additional transcriptomics machine learning model.

11. The system of claim 8, further comprising instructions that, when executed by the at least one processor, cause the system to:
generate adjusted observed transcriptomic profiles by modifying the observed transcriptomic profiles with the control transcriptomic profiles of the batches; and
generate adjusted predicted transcriptomic profiles by modifying the predicted transcriptomic profiles with predicted control transcriptomic profiles of the batches generated from the control transcriptomic profiles.

12. The system of claim 11, further comprising instructions that, when executed by the at least one processor, cause the system to generate the structural distance by comparing the adjusted observed transcriptomic profiles and the adjusted predicted transcriptomic profiles.

13. The system of claim 8, further comprising instructions that, when executed by the at least one processor, cause the system to determine a threshold structural distance based on a number of measured genes in the observed transcriptomic profiles and a number of samples in a batch.

14. The system of claim 13, further comprising instructions that, when executed by the at least one processor, cause the system to generate the structural integrity metric by comparing the structural distance and the threshold structural distance.

15. A non-transitory computer-readable medium storing instructions that, when executed by at least one processor, cause a computing device to:
generate, utilizing a transcriptomics machine learning model, transcriptomic embeddings from observed transcriptomic profiles of cells exposed to perturbations;
generate, utilizing the transcriptomic embeddings, a plurality of transcriptomic evaluation metrics comprising a structural integrity metric by:
reconstructing, utilizing a neural network, predicted transcriptomic profiles for the cells exposed to the perturbations from the transcriptomic embeddings; and
generating a structural distance for the transcriptomics machine learning model from the predicted transcriptomic profiles and the observed transcriptomic profiles relative to control transcriptomic profiles across batches; and
combine the plurality of transcriptomic evaluation metrics comprising the structural integrity metric to generate a transcriptomic benchmark for evaluating the transcriptomics machine learning model.

16. The non-transitory computer-readable medium of claim 15, further comprising instructions that, when executed by the at least one processor, cause the computing device to generate the plurality of transcriptomic evaluation metrics by:
generating a batch effect metric by comparing sets of transcriptomic embeddings across batches within an embedding feature space; and
generating at least one of: a latent space linear separability metric; a perturbation consistency metric; a latent space direct organization metric; or a zero-shot retrieval metric.

17. The non-transitory computer-readable medium of claim 15, further comprising instructions that, when executed by the at least one processor, cause the computing device to:
generate adjusted observed transcriptomic profiles by modifying the observed transcriptomic profiles with the control transcriptomic profiles of the batches; and
generate adjusted predicted transcriptomic profiles by modifying the predicted transcriptomic profiles with predicted control transcriptomic profiles of the batches generated from the control transcriptomic profiles.

18. The non-transitory computer-readable medium of claim 17, further comprising instructions that, when executed by the at least one processor, cause the computing device to generate the structural distance by comparing the adjusted observed transcriptomic profiles and the adjusted predicted transcriptomic profiles.

19. The non-transitory computer-readable medium of claim 15, further comprising instructions that, when executed by the at least one processor, cause the computing device to determine a threshold structural distance based on a number of measured genes in the observed transcriptomic profiles and a number of samples in a batch.

20. The non-transitory computer-readable medium of claim 19, further comprising instructions that, when executed by the at least one processor, cause the computing device to generate the structural integrity metric by comparing the structural distance and the threshold structural distance.
